(19) United States
(12) Patent Application Publication
(10) Pub. No.: US 2023/0149349 A1
(43) Pub. Date: May 18, 2023

(54) MEDICAMENT CONTAINING SOFPIRONIUM BROMIDE

(71) Applicant: KAKEN PHARMACEUTICAL CO., LTD., Tokyo (JP)

(72) Inventors: Hiroshi ISEKI, Tokyo (JP); Akihiro ONO, Shizuoka (JP); Daiki TSUBOI, Shizuoka (JP); Yuki NISHIHARA, Shizuoka (JP); Nobuyuki KODERA, Tokyo (JP); Toshiyuki YOROZUYA, Tokyo (JP); Motoki AKAMATSU, Tokyo (JP); Ippei OTANI, Tokyo (JP)

(73) Assignee: KAKEN PHARMACEUTICAL CO., LTD., Tokyo (JP)

(21) Appl. No.: 17/908,163

(22) PCT Filed: Mar. 2, 2021
(86) PCT No.: PCT/JP2021/007790

§ 371 Date: Aug. 30, 2022

(30) Foreign Application Priority Data
Mar. 3, 2020 (JP) 2020-035827
Sep. 2, 2020 (JP) 2020-147340

(51) Int. Cl. A61K 31/40; A61K 9/00; A61K 47/10; A61P 17/00; A61K 47/12; A61K 47/02

(52) U.S. Cl. CPC A61K 31/40; A61K 9/0014; A61K 47/10; A61P 17/00; A61K 47/12; A61K 47/02

(57) ABSTRACT

A pharmaceutical formulation for applying as an external application to the surface of the human body, in which decrease of viscosity during long-term storage is suppressed, and which contains sofpironium bromide, a water-soluble polymer, and ethanol, has a pH of 5.2 or lower, and is a uniformly dispersed, and non-aqueous formulation or low water content formulation with a water content of 5% or lower.

### TECHNICAL FIELD

The present invention relates to a pharmaceutical formulation containing sofpironium bromide as an active ingredient.

### BACKGROUND ART

Pharmaceutical formulations are required to be provided in a physicochemically stable form, and it is desirable that the properties, pharmaceutical characteristics, contents of related substances, and purities of the active ingredients thereof remain within certain ranges over a long period of time. In particular, in the case of formulations for topical administration to be externally applied, they are desirably formulations of which pharmaceutical characteristics are stable over a long period of time from viewpoints of handling and feeling of use.

Acetylcholine is known as one of the major neurotransmitters in the living bodies, and has a variety of pharmacological actions. For example, its perspiration activity based on activation of sweat glands is one of such actions. Therefore, anticholinergic agents are useful for the medical cares, therapeutic treatments, or preventions of various diseases related to acetylcholine such as hyperhidrosis.

Hyperhidrosis is a pathological condition in which excessive sweating occurs in the palms, soles, and axillae due to heat, mental stress, or other causes, causing troubles in daily life (for example, paper documents and notebooks are torn by sweat, patients cannot join hands with others due to the concern about sweat, underwear needs to be changed many times a day, mobile phones are wet and broken by sweat, and the like), and markedly impairing QOL (Non-patent document 1). Human sweat glands include eccrine sweat glands and apocrine sweat glands, and the sweat that causes hyperhidrosis is secreted by the eccrine sweat glands (Non-patent document 2). The eccrine sweat glands are regulated by cholinergic nerves, and it is thought that acetylcholine induces sweating by stimulating M3 muscarinic receptors locating in the postsynaptic membrane of eccrine sweat glands (Non-patent document 3).

Hyperhidrosis is classified into generalized hyperhidrosis and focal hyperhidrosis depending on whether the affected area is systemic or part of the body, and focal hyperhidrosis often occurs in the palms, soles, and axillae. Hyperhidrosis can also be classified into primary hyperhidrosis with no specific etiology, and secondary hyperhidrosis associated with other diseases (e.g., use of drugs, cardiovascular diseases and the like are involved in the generalized hyperhidrosis, and peripheral neuropathy and the like are involved in the focal hyperhidrosis). As described above, the primary axillary hyperhidrosis is a pathological condition in which excessive sweating occurs in the axillae without a specific etiology, and which interferes with daily life.

Anticholinergic agents for external application that are useful in the therapeutic treatment of hyperhidrosis include soft glycopyrrolates (Patent document 1). Soft glycopyrrolates are derivatives of glycopyrrolate, which is an anticholinergic agent, and one of the typical soft glycopyrrolates is sofpironium bromide.

Sofpironium bromide is an ester compound represented by the following formula (I):

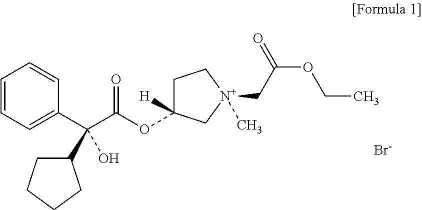

and epimer at N⁺ (hereinafter also referred to as “BBI-4000” or “compound (I)”), and is a bromide salt of quaternary ammonium. Various external formulations for externally applying sofpironium bromide have been reported so far.

Patent document 2 discloses a formulation for external application containing BBI-4000, ethanol, Dimethiconol Blend 20, and Klucel (registered trademark, hydroxypropylcellulose, hereinafter also referred to as “HPC”) (e.g., Table III), and reports that this formulation can be used for the therapeutic treatment of hyperhidrosis.

Patent documents 3 and 4 describe that in formulations containing Dimethiconol Blend 20, a small amount of Dimethiconol Blend 20 coalesces as small droplets at the bottom of the container over time, and discloses a formulation containing BBI-4000, ethanol, isopropyl myristate (hereinafter, also referred to as “IPM”), and hydroxypropylcellulose (HPC) as a formulation that does not generate droplets (e.g., TABLE VIII).

In designing external formulations, the viscosity of the formulations is one of the important physical properties because it influences the retentivity of the active ingredient on affected area. If appropriate formulation viscosity is not maintained, the medicament cannot be retained on the affected area, which influences the patients' feeling of use due to dripping, adhering to clothes, and the like. Therefore, it is necessary to develop a stable external formulation that provides excellent patients' feeling of use, and does not show significant change in the characteristics thereof such as viscosity and spreadability even after long-term storage.

In general, water-soluble polymers such as cellulose polymers are added to external formulations to impart viscosity and other properties. However, the viscosity of external formulations imparted by water-soluble polymers may decrease over time due to degradation of the polymers by light or heat. In particular, when cellulose polymers are blended into high water content formulations, the viscosity of the formulations may decrease over time because of the low viscosity stability. On the other hand, for non-aqueous or low water content formulations containing water-soluble polymers, the phenomenon of viscosity decrease over time has not been reported so far, and there is no previous knowledge as to under what conditions viscosity decrease over time can be suppressed.

Patent documents 2, 3, and 4 mentioned above disclose non-aqueous formulations containing sofpironium bromide and a water-soluble polymer, but they neither disclose nor suggest any means for imparting high stability that enables long-term storage. They also neither disclose nor suggest any highly stable low water content formulation containing sofpironium bromide and a water-soluble polymer. Even more, any means for maintaining viscosity of a non-aqueous or low water content formulation containing sofpironium bromide and a water-soluble polymer is not known at all.

### PRIOR ART REFERENCES

### Patent Documents

Patent document 1: International Publication WO2014/144075
Patent document 2: International Publication WO2015/138776
Patent document 3: International Publication WO2017/015485
Patent document 4: International Publication WO2018/017852

### Non-Patent Documents

Non-patent document 1: Journal of the Japanese Dermatological Association, 2015; 125: 1379-1400
Non-patent document 2: Development and structure of sweat glands, MB Derma, 2014; 220: 9-12
Non-patent document 3: Hyperhidrosis—Causes and treatment of enhanced sweating, Dtsch Arztebl Int., 2009; 106: 32-7

### DISCLOSURE OF THE INVENTION

### Object to be Achieved by the Present Invention

An object to be achieved by the present invention is to provide a means for suppressing decrease of viscosity of a non-aqueous or low water content formulation for applying as an external application containing sofpironium bromide as an active ingredient during long-term storage.

Another object to be achieved by the present invention is to provide a non-aqueous or low water content formulation of sofpironium bromide for external application, in which decrease of viscosity is suppressed over long-term storage, and which provides no change in patients' feeling of use, and has stable formulation characteristics as a pharmaceutical product.

A further object to be achieved by the present invention is to provide an external formulation of sofpironium bromide that shows therapeutic effects on diseases in which acetylcholine is involved (e.g., primary focal hyperhidrosis, and the like).

### Means for Achieving the Objects

The inventors of the present invention conducted studies on external formulations of sofpironium bromide stable over a long time of period, and as a result, they revealed that, in non-aqueous formulations of sofpironium bromide, the viscosity of the formulations imparted by water-soluble polymers decreases over time.

As described above, there had been no report of viscosity decrease over time in non-aqueous or low water content formulations containing water-soluble polymers, and it was completely unexpected that such a phenomenon of viscosity decrease over time would occur in non-aqueous formulations of sofpironium bromide. Since decrease in the viscosity of the formulations over time affects the patients' feeling of use, and the like, it is desirable to avoid this problem. Therefore, the inventors of the present invention conducted an intensive study to avoid the problem, which had not been known till then in this field.

As a result of detailed studies on factors affecting the stability of sofpironium bromide formulations, the inventors of the present invention revealed that pH of non-aqueous formulations containing sofpironium bromide and a water-soluble polymer significantly affects the stability of the formulations, and that by maintaining the pH of the formulations at 5.2 or lower, the decrease in viscosity over time can be suppressed.

Then, the inventors of the present invention studied in detail the effect of water content in the sofpironium bromide formulations on the stability of the formulations. From technical common sense of those skilled in the art, it was expected that the stability of the formulations would be impaired if the water content was increased. However, surprisingly, it became clear that even in low water content formulations with a water content of 5% or lower, if the pH of the formulations is maintained at 5.2 or lower, increase in relates substances is negligible, and the decrease in viscosity over time can also be suppressed. Furthermore, the inventors of the present invention also found that the aforementioned viscosity decrease suppressing effect is independent of the types of additives such as non-volatile oil and pH adjuster, and that a physicochemically stable sofpironium bromide formulation can be obtained by maintaining the pH of the formulation at 5.2 or lower.

The inventors of the present invention further conducted the studies and found that the formulation mentioned above is stable for a long period of time and has excellent characteristics as a pharmaceutical product, and the formulation is an extremely effective to be applied in clinical practice, and thus accomplished the present invention.

The present invention thus includes the following inventions.

A pharmaceutical formulation for applying as an external application to the surface of the human body, wherein the formulation contains:

(a) sofpironium bromide,
(b) one or more kinds of water-soluble polymers, and
(c) ethanol,
wherein the formulation has a pH of 5.2 or lower and is a uniformly dispersed, and non-aqueous formulation or low water content formulation with a water content of 5 w/w % or lower, and
wherein the pH is measured at any one or more time points selected within 6 months after preparation of the formulation, the pH is determined by measuring pH of the formulation stored at room temperature, and the pH is a value obtained after a pH electrode for non-aqueous solvent is immersed in the formulation for 5 minutes.

The formulation according to [01] mentioned above, wherein content of sofpironium bromide is 1 to 20 w/w % based on the total weight of the formulation.

The formulation according to [01] or [02] mentioned above, wherein the pH is in the range of 2.5 to 5.2.

The formulation according to [01] or [02] mentioned above, wherein ingredients in the formulation are uniformly dissolved.

The formulation according to any one of [01] to [04] mentioned above, wherein the water-soluble polymer or polymers consist of a water-soluble vinyl polymer or a water-soluble cellulose polymer.

The formulation according to any one of [01] to [04] mentioned above, wherein the water-soluble polymer or polymers are selected from the group consisting of hydroxymethylcellulose, hydroxyethylcellulose, hydroxypropylcellulose, hydroxyethylmethylcellulose, hydroxypropylmethylcellulose, methylcellulose, ethylcellulose, carboxymethylcellulose, a carboxyvinyl polymer, polyvinyl alcohol, a polyvinyl copolymer, polyvinylpyrrolidone, and copovidone.

The formulation according to any one of [01] to [04] mentioned above, wherein the water-soluble polymer or polymers consist of hydroxypropylcellulose or a carboxyvinyl polymer.

The formulation according to any one of [01] to [07] mentioned above, wherein content of the water-soluble polymer or polymers is 0.01 to 5.0 w/w % based on the total weight of the formulation.

The formulation according to any one of [01] to [08] mentioned above, wherein content of ethanol is 50 to 99 w/w % based on the total weight of the formulation.

The formulation according to any one of [01] to [09] mentioned above, further containing a pH adjuster.

The formulation according to [10] mentioned above, wherein the pH adjuster is an acid selected from the group consisting of tartaric acid, acetic acid, and citric acid, or a salt thereof.

The formulation according to [10] or [11] mentioned above, wherein the amount of the pH adjuster is 0.015 to 5 w/w % based on the total weight of the formulation.

The formulation according to any one of [01] to [12] mentioned above, further containing a non-volatile oil (except for formulation containing Dimethiconol Blend 20).

The formulation according to [13] mentioned above, wherein the non-volatile oil is selected from the group consisting of a non-volatile ester, a non-volatile ether, a non-volatile silicone, and a non-volatile alcohol.

The formulation according to [13] mentioned above, wherein the non-volatile oil is a non-volatile ester selected from the group consisting of a monoester, a diester, and a trimester, and wherein the non-volatile oil is represented as R₁COOR₂, wherein one of R₁ and R₂ is a C₄-C₄₀ straight chain alkyl group that may be substituted, or a C₄-C₄₀ branched chain alkyl group that may be substituted, and the other of R₁ and R₂ is a C₁-C₄₀ alkyl group that may be substituted.

The formulation according to [13] mentioned above, wherein the non-volatile oil is a non-volatile fatty acid ester selected from the group consisting of ethyl myristate, 2-octyldodecyl myristate, butyl stearate, isocetyl stearate, 2-octyldodecyl stearate, hexyl laurate, 2-hexyldecyl laurate, 2-ethylhexyl palmitate, 2-octyldecyl palmitate, cetearyl octanoate, isononyl isononanoate, octyldodecyl neopentanoate, 2-octyldodecyl erucate, 2-octyldodecyl benzoate, a decanoic acid ester, a ricinoleic acid ester, isopropyl myristate, diisopropyl adipate, a medium chain fatty acid triglyceride, isopropyl palmitate, an alkyl (C₁₄-C₁₈) ethylhexanoate, myristyl myristate, ethyl oleate, oleyl oleate, ethylhexyl palmitate, cetyl palmitate, 2-hexyldecyl myristate, 2-hexyldecyl palmitate, PPG-3 benzyl ether myristate, isotridecyl isononanoate, triethylhexyl trimellitate, an alkyl (C₁₂-C₁₅) benzoate, diethoxyethyl succinate, propylene glycol dicaprate, propylene glycol dicaprylate, glyceryl tri(caprylate/caprate), triethylhexanoin, triisostearin, isopropyl isostearate, isostearyl isostearate, polyglyceryl-2 triisostearate, diethylhexyl succinate, PPG-2 myristyl propionate, pentaerythrityl tetraisostearate, diethyl sebacate, PPG-3 benzyl ether ethylhexanoate, glyceryl tribehenate, cetyl 2-ethylhexanoate, diisostearyl malate, 2-ethylhexyl stearate, triethylhexyl citrate, and an alkyl lactate such as ethyl lactate.

The formulation according to [13] mentioned above, wherein the non-volatile oil is a non-volatile fatty acid ester selected from the group consisting of isopropyl myristate, diisopropyl adipate, and a medium chain fatty acid triglyceride.

The formulation according to [13] mentioned above, wherein the non-volatile oil is a non-volatile silicone selected from the group consisting of a medical grade silicone oil, methylphenyl silicone, methyl terminated hydrogen branched silicone, decamethyl pentacyclosiloxane, octamethyl tetracyclosiloxane, cyclomethicone 5-NF, PEG-12 dimethicone, dimethicone 20 cSt, dimethicone 100 cSt, dimethicone 350 cSt, dimethicone 500 cSt, dimethicone 1000 cSt, and dimethicone 12500 cSt.

The formulation according to [13] mentioned above, wherein the non-volatile oil is a non-volatile silicone selected from the group consisting of cyclomethicone 5-NF, PEG-12 dimethicone, dimethicone 20 cSt, and dimethicone 350 cSt.

The formulation according to any one of [13] to [19] mentioned above, wherein content of the non-volatile oil is 0.5 to 10 w/w % based on the total weight of the formulation.

The formulation according to any one of [01] to [20] mentioned above, further containing a polyhydric alcohol.

The formulation according to [21] mentioned above, wherein the polyhydric alcohol is selected from the group consisting of hexylene glycol, propylene glycol, ethylene glycol, glycerol, and butylene glycol.

The formulation according to [21] or [22] mentioned above, wherein content of the polyhydric alcohol is 1.0 to 30 w/w % based on the total weight of the formulation.

The formulation according to any one of [01] to [23] mentioned above, wherein viscosity of the formulation is 10 to 2000 mPa·s at 25° C.

The formulation according to any one of [01] to [24] mentioned above, wherein viscosity of the formulation is 10 to 1000 mPa·s at 25° C. after storage at room temperature for 36 months from preparation of the formulation or after storage at 40° C. for 3 months from preparation of the formulation.

The formulation according to any one of [01] to [25] mentioned above, which is for medical care, therapeutic treatment, or prevention of a disease selected from the group consisting of hyperhidrosis, overactive bladder, chronic obstructive pulmonary disease, cardiac disease, salivation, ocular disease, and bronchial asthma.

A formulation for applying as an external application, wherein content of a compound (II) represented by the following formula (II):

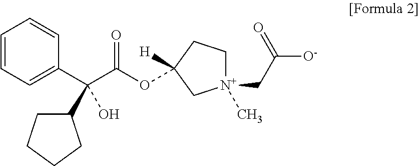

and epimer at N⁺
is 1.5 w/w % or lower based on the content of sofpironium bromide, and purity of sofpironium bromide is 90 w/w % or higher after storage at room temperature for 36 months from preparation of the formulation, or after storage at 40° C. for 3 months from preparation of the formulation.

The formulation according to any one of [01] to [27] mentioned above for applying as an external application for medical care, therapeutic treatment, or prevention of primary axillary hyperhidrosis in which total sweating weight in both axillae for 5 minutes before treatment is 100 mg or more as determined by the gravimetric method, characterized by containing sofpironium bromide as an active ingredient, and being topically administered to both axillae once a day.

The formulation according to [28] mentioned above for medical care, therapeutic treatment, or prevention of primary axillary hyperhidrosis in which total sweating weight in both axillae for 5 minutes before treatment is 400 mg or more as determined by the gravimetric method characterized by being externally applied over a treatment period of at least 6 weeks.

### Effect of the Invention

According to the present invention, by maintaining pH of a non-aqueous or low water content formulation containing sofpironium bromide and a water-soluble polymer at 5.2 or lower, decrease in viscosity of the formulation over time during long-term storage can be suppressed, and thus a formulation for applying as an external application having superior characteristics as a pharmaceutical composition can be provided.

### BRIEF DESCRIPTION OF THE DRAWINGS

FIG. 1 Time course of Test Example 7: Confirmatory study of BBI-4000 using patients with primary axillary hyperhidrosis as the subjects. In the FIGURE, *1 the span of the baseline includes the time points of performing three times of measurements for baselines 1 to 3, and *2 the span of the end of the treatment includes the time points of performing three times of measurements for week 6 of administration 1 to 3.

### MODE FOR CARRYING OUT THE INVENTION

Hereafter, the present invention is explained in detail.

The formulation of the present invention is a formulation for topical administration containing sofpironium bromide as an active ingredient.

The content of sofpironium bromide in the formulation of the present invention is not particularly limited, but is preferably 1 to 30 w/w %, more preferably 1 to 20 w/w %, still further preferably 5 to 15 w/w %.

In one embodiment of the present invention, a particularly preferred sofpironium bromide content is 5 w/w % based on the total weight of the formulation.

In another embodiment of the present invention, a particularly preferred content of sofpironium bromide is 10 w/w % based on the total weight of the formulation.

In another embodiment of the present invention, a particularly preferred content of sofpironium bromide is 15 w/w % based on the total weight of the formulation.

In this description, when a range is described as “A to B”, “A-B”, “A˜B”, or the like, the range includes the numerical values at the ends of the range unless especially noted.

The formulation of the present invention is not particularly limited so long as it is a pharmaceutical formulation for applying as an external application to the surface of human body, and may be a liquid, lotion, ointment, cream, or gel preparation.

The formulation of the present invention is preferably a liquid or gel preparation, more preferably a liquid preparation.

The formulation of the present invention contains sofpironium bromide as the active ingredient, and can be used as a medicament for medical care, therapeutic treatment, or prevention of various diseases related to the action of acetylcholine by topically administering it to the human body surface.

The term “body surface” used in this description refers to the surface of human skin, and the like. Specifically, the term “body surface” refers to the skin surfaces of the extremities, body parts, and head, more specifically, skin surfaces of the palm, head, face, shoulder, chest, buttock, abdomen, back, pubic region, axilla, and the like, hair, nail, and the like. According to one embodiment of the present invention, body surfaces (application sites) suitable for the application are not particularly limited, but skin surfaces are preferred, and for example, skin surfaces of axillae are particularly preferred.

The term “topical administration” or “external application” used in this description means application of a pharmaceutical formulation to a lesion on a human body surface or its surrounding area.

In one embodiment of the present invention, the formulation of the present invention is a liquid preparation for external use for applying a medicament to the axilla.

In another embodiment of the present invention, the formulation of the present invention is a liquid preparation for external use for applying a medicament to the palm.

In another embodiment of the present invention, the formulation of the present invention is a liquid preparation for external use for application of a medicament to the body.

In general, primary focal hyperhidrosis causes excessive sweating symmetrically on the head, face, palms, soles, axillae, and the like. Therefore, the formulation of the present invention is preferably a formulation for external use for application to both axillae or both palms, but if excessive sweating occurs in one axilla or one palm, and the like, the formulation of the present invention can also be applied to the one axilla or one palm.

In this description, the term “uniformly dispersed” means that the composition of the formulation is homogenous, equilibrium, and stable. Specifically, it means that under normal storage conditions (for example, for a storage period of 3 years at room temperature, and the like), it does not show separation of liquid phases, generation of droplets, and precipitation of prescribed ingredient or other ingredients, and examples of a formulation in such a state includes, for example, a formulation in which ingredients are uniformly dissolved.

The formulation of the present invention is a formulation of which constituent ingredients are uniformly dispersed, and oil droplets and the like are not generated, and it can be stably stored under normal storage conditions.

The formulation of the present invention is preferably a formulation in which ingredients thereof are uniformly dissolved, more preferably a clear formulation in which ingredients thereof are uniformly dissolved.

Further, the formulation of the present invention does not cause discoloration, alteration, large increase or decrease in the content of the active ingredient, large increase in related substances or microbiological quality problems over time, which may deviate the formulation from the pharmaceutical formulation specifications, and thus it is desirable as a pharmaceutical formulation.

In this description, the term “water content” means weight ratio of contained water to the total weight of the formulation.

In this description, “non-aqueous formulation” means a formulation in which water content in the formulation is 0 w/w % or a formulation substantially free from water.

In this description, “substantially water-free formulation” means, for example, a formulation in which water content in the formulation is 1 w/w % or lower.

In this description, “low water content formulation” means a formulation in which water content in the formulation is 20 w/w % or lower.

In one embodiment of the present invention, the water content of the formulation of the present invention is preferably 10 w/w % or lower, more preferably 5 w/w % or lower, further preferably 3 w/w % or lower, still further preferably 2 w/w % or lower, particularly preferably 1 w/w % or lower.

In one embodiment of the present invention, the water content of the formulation of the present invention is preferably 0.001 to 10 w/w %, more preferably 0.001 to 5 w/w %, further preferably 0.001 to 3 w/w %.

In another embodiment of the present invention, the formulation of the present invention is preferably a non-aqueous formulation or a low water content formulation with a water content of 5 w/w % or lower, more preferably a non-aqueous formulation or a low water content formulation with a water content of 3 w/w % or lower, further preferably a non-aqueous formulation or a low water content formulation with a water content of 2 w/w % or lower, still further preferably a non-aqueous formulation or a low water content formulation with a water content of 1 w/w %, particularly preferably a non-aqueous formulation.

In another embodiment of the present invention, the formulation of the present invention is preferably a non-aqueous formulation or a low water content formulation with a water content of 0.001 to 5 w/w %, more preferably a non-aqueous formulation or a low water content formulation with a water content of 0.001 to 3 w/w %, further preferably a non-aqueous formulation or a low water content formulation with a water content of 0.001 to 2 w/w %, still further preferably a non-aqueous formulation or a low water content formulation with a water content of 0.001 to 1 w/w %, most preferably a non-aqueous formulation.

In another embodiment of the present invention, the formulation of the present invention is preferably a low water content formulation with a water content of 0.001 to 5 w/w %, more preferably a low water content formulation with a water content of 0.002 to 3 w/w %, further preferably a low water content formulation with a water content of 0.005 to 2 w/w %, still further preferably a low water content formulation with a water content of 0.01 to 1 w/w %.

The water-soluble polymer contained in the formulation of the present invention is not particularly limited so long as it is a water-soluble polymer that can be used as additive in pharmaceuticals and can impart a certain degree of viscosity to the formulation.

The water-soluble polymer is preferably a water-soluble polymer that gives a viscosity of 2.0 to 2000 mPa·s at 25° C. to a non-aqueous formulation when the formulation contains 1.25 w/w % of the water-soluble polymer based on the total weight of the formulation. The water-soluble polymer is preferably a water-soluble polymer that gives a viscosity of 5.0 to 1500 mPa·s under the same conditions, further preferably a water-soluble polymer that gives a viscosity of 10 to 1000 mPa·s under the same conditions, still further preferably a water-soluble polymer that gives a viscosity of 100 to 800 mPa·s under the same conditions.

Specific examples of the water-soluble polymer include cellulose polymers, vinyl polymers, and acrylate polymers.

Specific examples of the cellulose polymers include hydroxyalkylcelluloses (e.g., hydroxymethylcellulose (HMC), hydroxyethylcellulose (HEC), hydroxypropylcellulose (HPC), and hydroxybutylcellulose), hydroxyalkylalkyl celluloses (e.g., hydroxyethylmethylcellulose (HEMC), and hydroxypropylmethylcellulose (HPMC)), alkylcelluloses (e.g., methylcellulose), carboxymethylcellulose, cellulose esters (cellulose acetate), and the like.

Specific examples of the vinyl polymers include carboxyvinyl polymers, polyvinyl alcohol, polyvinyl copolymers (copolymers in which polyvinyl alcohol is one of the monomers, such as polyvinyl alcohol-acrylic acid-methyl methacrylate copolymers, polyvinyl alcohol-polyethylene glycol graft copolymers, polyvinyl caprolactam-polyvinyl acetate-polyethylene glycol graft copolymers, and the like), polyvinyl pyrrolidone (povidone), copovidone, vinyl acetate resin, and the like.

Specific examples of the acrylate polymers include aminoalkyl methacrylate copolymers (e.g., aminoalkyl methacrylate copolymer RS), ethyl acrylate-methyl methacrylate copolymers, and the like.

In the present invention, preferred water-soluble polymers are cellulose polymers.

In one embodiment of the present invention, the cellulose polymer is preferably HMC, HEC, HPC, HEMC, HPMC, methylcellulose, ethylcellulose, or carboxymethylcellulose, further preferably HEC, HPC, or HPMC, still further preferably HPC.

In another embodiment of the present invention, the cellulose polymer is preferably hydroxyalkylcellulose or hydroxyalkylalkylcellulose, more preferably hydroxy(C₂-C₄ alkyl)cellulose or hydroxy(C₂-C₄ alkyl)(C₁-C₄ alkyl)cellulose, further preferably hydroxy(C₂-C₄ alkyl)cellulose.

“Hydroxyalkylcellulose” refers to cellulose in which hydroxyl groups of cellulose are substituted by a number of hydroxyalkyl groups, which is a reaction product of cellulose and an alkylene oxide such as ethylene oxide and propylene oxide.

“Hydroxy(C₂-C₄ alkyl)cellulose” refers to hydroxyalkylcellulose with hydroxyalkyl groups having 2 to 4 carbon atoms. Specific examples thereof include hydroxyethylcellulose (HEC), hydroxypropylcellulose (HPC), and the like.

“Hydroxyalkylalkylcellulose” refers to cellulose in which hydroxyl groups of cellulose are substituted by a large number of alkyl and hydroxyalkyl groups. Hydroxy(C₂-C₄ alkyl)(C₁-C₄ alkyl)cellulose” refers to hydroxyalkylalkylcellulose in which hydroxyalkyl groups have 2 to 4 carbon atoms, and alkyl groups have 1 to 4 carbon atoms. Specific examples thereof include hydroxypropylmethylcellulose (HPMC), and the like.

In one embodiment of the present invention, the content of the water-soluble polymer is not particularly limited, but it is preferably 0.01 to 5.0 w/w %, more preferably 0.1 to 2.5 w/w %, further preferably 0.5 to 2.0 w/w %, still further preferably 1.0 to 1.5 w/w %, particularly preferably 1.25 w/w %, based on the total weight of the formulation.

In one embodiment of the present invention, the water-soluble polymer is preferably 0.01 to 5.0 w/w % of HEC, HPC, or HPMC, more preferably 0.1 to 2.5 w/w % of HEC, HPC, or HPMC, further preferably 0.5 to 2.0 w/w % of HEC, HPC, or HPMC, still further preferably 1.0 to 1.5 w/w % of HEC, HPC, or HPMC, particularly preferably 1.25 w/w % of HEC, HPC, or HPMC, even more preferably 1.0 w/w % or more and 1.5 w/w % or less of HEC, HPC, or HPMC, especially preferably 1.25 w/w % of HEC, HPC, or HPMC, based on the total weight of the formulation.

In one embodiment of the present invention, the formulation of the present invention contains ethanol as a solvent.

In this description, “ethanol” is a term that encompasses various grades of ethanol, for example, anhydrous ethanol and 95% ethanol.

In one embodiment of the present invention, ethanol is preferably 95% ethanol.

In another embodiment of the present invention, ethanol is preferably anhydrous ethanol.

In one embodiment of the present invention, the content of ethanol is preferably 30 to 95 w/w %, more preferably 50 to 90 w/w %, further preferably 60 to 85 w/w %, still further preferably 70 to 85 w/w %, based on the total weight of the formulation.

In another embodiment of the present invention, the content of ethanol is preferably 60 to 95 w/w %, more preferably from 60 to 90 w/w %, further preferably 60 to 85 w/w %, based on the total weight of the formulation.

In this description, pH refers to a value thereof measured after immersing a pH electrode for non-aqueous solvent in a test formulation for 5 minutes.

The pH electrode for non-aqueous solvent can be, for example, a pH electrode for low conductivity water and non-aqueous solvent (a pH electrode that can be used for both low conductivity water and non-aqueous solvents).

In this description, for example, pH refers a value thereof measured after immersing a pH electrode for low conductivity water and non-aqueous solvent, calibrated with a pH standard solution, in 10.0 g of a test formulation for 5 minutes.

The pH measurement using a pH electrode for low conductivity water and non-aqueous solvent is described in the Japanese Pharmacopoeia, Seventeenth Edition (Hirokawa Shoten), and the measurements described in this description were performed in accordance with the Japanese Pharmacopoeia. ApH electrode for low conductivity water and non-aqueous solvent can be readily obtained as a commercial product from, for example, HORIBA Advanced Techno. The pH measurement can be performed by using the above electrode at a temperature of 0 to 60° C., preferably 1 to 30° C., still further preferably 20 to 30° C.

The expression “immersing a pH electrode” used here refers to immersing the electrode so that the liquid junction of the pH electrode is completely immersed in the test formulation, and thus pH can be accurately measured.

In one embodiment of the present invention, the time point at which pH is measured in the present invention is not particularly limited. That is, if there is no special note regarding the time point of measurement, it can be pH measured at any time point, such as pH measured immediately after the preparation of the formulation, pH measured after 1 month from the preparation, pH measured after 2 months from the preparation, pH measured after 3 months from the preparation, pH measured after 6 months from the preparation, pH measured after 12 months from the preparation, pH measured after 24 months from the preparation, and pH measured after 36 months from the preparation.

In one embodiment of the present invention, the formulation of the present invention has a pH of 5.2 or lower at the time of the preparation of the formulation, a pH of 5.2 or lower after 1 month from the preparation, a pH of 5.2 or lower after 2 months from the preparation, a pH of 5.2 or lower after 3 months from the preparation, a pH of 5.2 or lower after 6 months from the preparation, a pH of 5.2 or lower after 12 months from the preparation, a pH of 5.2 or lower after 18 months from the preparation, a pH of 5.2 or lower after 24 months from the preparation, or a pH of 5.2 or lower after 36 months from the preparation.

In one embodiment of the present invention, the formulation of the present invention has a pH in the range of 2.5 to 5.2 at the time of the preparation of the formulation, a pH in the range of 2.5 to 5.2 after 1 month from the preparation, a pH in the range of 2.5 to 5.2 after 2 months from the preparation, a pH in the range of 2.5 to 5.2 after 3 months from the preparation, a pH in the range of 2.5 to 5.2 after 6 months from the preparation, a pH in the range of 2.5 to 5.2 after 12 months from the preparation, a pH in the range of 2.5 to 5.2 after 18 months from the preparation, a pH in the range of 2.5 to 5.2 after 24 months from the preparation, or a pH in the range of 2.5 to 5.2 after 36 months from the preparation.

In one embodiment of the present invention, when the formulation of the present invention is stored at room temperature, the formulation has a pH in the range of 2.5 to 5.2 at the time of the preparation of the formulation, a pH in the range of 2.5 to 5.2 after 1 month from the preparation, a pH in the range of 2.5 to 5.2 after 2 months from the preparation, a pH in the range of 2.5 to 5.2 after 3 months from the preparation, a pH in the range of 2.5 to 5.2 after 6 months from the preparation, a pH in the range of 2.5 to 5.2 after 12 months from the preparation, a pH in the range of 2.5 to 5.2 after 18 months from the preparation, a pH in the range of 2.5 to 5.2 after 24 months from the preparation, or a pH in the range of 2.5 to 5.2 after 36 months from the preparation.

In each of the above embodiments, it is sufficient if a pH value at any time point satisfies the above conditions, but it is preferred if pH values at two or more time points satisfy the conditions, and particularly preferred if pH values at all time points satisfy the above conditions.

In one embodiment of the present invention, the pH of the formulation of the present invention is measured at one or more time points selected within a period of 6 months from the preparation, and is a pH determined by measuring pH of the formulation stored at room temperature.

In one embodiment of the present invention, the pH of the formulation of the present invention is measured at one or more time points selected within a period of 6 months after the preparation, and is a pH determined by measuring pH of the formulation stored at room temperature from the preparation to the measurement.

In one embodiment of the present invention, the pH of the formulation of the present invention is measured at one or more time points selected within a period of 1 month after the preparation, and is a pH determined by measuring pH of the formulation stored at room temperature from the preparation to the measurement.

The pH of the formulation of the present invention is 5.2 or lower, preferably 2.5 to 5.2, more preferably 3.0 to 5.2, further preferably 3.0 to 5.0.

In one embodiment of the present invention, the pH measured at one or more time points selected within a period of 6 months from the preparation of the formulation, and determined by measuring pH of the formulation stored at room temperature from the preparation to the measurement is 5.2 or lower, preferably 2.5 to 5.2, more preferably 3.0 to 5.2, further preferably 3.0 to 5.0.

In this description, room temperature means 1 to 30° C.

The pH of the formulation of the present invention is maintained at 5.2 or lower, preferably 2.5 to 5.2.

In one embodiment of the present invention, the pH of the formulation of the present invention is maintained more preferably at 2.5 to 5.0, further preferably at 2.5 to 4.5.

In another embodiment of the present invention, the pH of the formulation of the present invention is maintained preferably at 3.0 to 5.2, more preferably at 3.0 to 5.0, further preferably at 3.0 to 4.5.

In one embodiment of the present invention, more typically, the pH of the formulation of the present invention refers to the highest pH value during the period in which the formulation is stored. For example, when it is stated that “the pH is 5.2 or lower”, it means that the highest value of the pH is 5.2 or lower during the period in which the formulation is stored, in other words, the pH is maintained at 5.2 or lower during the storage period.

In one embodiment of the present invention, when the formulation of the present invention is stored at 40° C. for 3 months after the preparation of the formulation, the pH of the formulation is maintained at 2.5 to 5.2, preferably 2.5 to 5.0, more preferably 2.5 to 4.5, for 3 months after the preparation of the formulation.

In another embodiment of the present invention, when the formulation of the present invention is stored at room temperature for 36 months after the preparation of the formulation, the pH of the formulation is maintained at 5.2 or lower, preferably 5.0 or lower, more preferably 4.8 or lower, for 36 months after the preparation of the formulation.

In another embodiment of the present invention, when the formulation of the present invention is stored at room temperature for 36 months after the preparation of the formulation, the pH of the formulation is maintained at 2.5 to 5.2, preferably 3.0 to 5.2, more preferably 3.0 to 5.0, for 36 months after the preparation of the formulation.

In another embodiment of the present invention, when the formulation of the present invention is stored at room temperature for 24 months after the preparation of the formulation, the pH of the formulation is maintained at 5.2 or lower, preferably 5.0 or lower, more preferably 4.8 or lower, for 24 months after the preparation of the formulation.

In another embodiment of the present invention, when the formulation of the present invention is stored at room temperature for 24 months after the preparation of the formulation, the pH of the formulation is maintained at 2.5 to 5.2, preferably 3.0 to 5.2, more preferably 3.0 to 5.0, for 24 months after the preparation of the formulation.

In another embodiment of the present invention, when the formulation of the present invention is stored at room temperature for 12 months after the preparation of the formulation, the pH of the formulation is maintained at 5.2 or lower, preferably 5.0 or lower, more preferably 4.8 or lower, for 12 months after the preparation of the formulation.

In another embodiment of the present invention, when the formulation of the present invention is stored at room temperature for 12 months after the preparation of the formulation, the pH of the formulation is maintained at 2.5 to 5.2, preferably 3.0 to 5.2, more preferably 3.0 to 5.0, for 12 months after the preparation of the formulation.

In another embodiment of the present invention, when the formulation of the present invention is stored at room temperature for 6 months after the preparation of the formulation, the pH of the formulation is maintained at 5.2 or lower, preferably 5.0 or lower, more preferably 4.8 or lower, for 6 months after the preparation of the formulation.

In another embodiment of the present invention, when the formulation of the present invention is stored at room temperature for 6 months after the preparation of the formulation, the pH of the formulation is maintained at 2.5 to 5.2, preferably 3.0 to 5.2, more preferably 3.0 to 5.0, for 6 months after the preparation of the formulation.

In another embodiment of the present invention, when the formulation of the present invention is stored at 40° C. for 3 months after the preparation of the formulation, the pH of the formulation is maintained at 3.0 to 5.2, preferably 3.0 to 5.0, more preferably 3.0 to 4.8, further preferably 3.0 to 4.5, for 3 months after the preparation of the formulation.

The formulation of the present invention may further contain a pH adjuster to maintain the pH within any of the preferred ranges mentioned above. The type of the pH adjuster is not particularly limited so long as a pH adjuster can be used as an additive in pharmaceutical products, and includes, for example, inorganic acids, inorganic acid salts, organic acids, organic acid salts, and the like.

Inorganic acids include, for example, hydrochloric acid, sulfuric acid, phosphoric acid, nitric acid, hydrobromic acid, hydroiodic acid, and the like.

Inorganic acid salts include, for example, ammonium hydrochloride, potassium carbonate, sodium monohydrogenphosphate, sodium dihydrogenphosphate, and the like.

Organic acid means an acid having at least one carbon atom in the chemical structure thereof, and typically refers to a monovalent organic acid, divalent organic acid, or trivalent organic acid.

Specific examples of organic acids include organic carboxylic acids such as acetic acid, propionic acid, trifluoroacetic acid, benzoic acid, maleic acid, fumaric acid, succinic acid, tannic acid, butyric acid, valeric acid, hibenzoic acid, pamoic acid, enanthic acid, tartronic acid, decanoic acid, theocic acid, salicylic acid, α-hydroxy acids, amino acids, and oxalic acid, organic sulfonic acids such as methanesulfonic acid, benzenesulfonic acid, and p-toluenesulfonic acid, and the like.

Specific examples of α-hydroxy acids include glycolic acid, L-lactic acid, DL-lactic acid, D-lactic acid, malic acid, citric acid, L-tartaric acid, DL-tartaric acid, D-tartaric acid, mandelic acid, arabic acid, gluconic acid, and the like.

Specific examples of amino acids include glycine, alanine, glutamic acid, aspartic acid, phenylalanine, β-alanine, isoleucine, leucine, proline, glutamine, serine, threonine, valine, tryptophan, tyrosine, and the like.

“Citric acid” mentioned in this description may also be anhydrous citric acid, citric acid hydrate, or the like.

The pH adjusters listed above may be any of their stereoisomers, geometric isomers, hydrates, anhydrates, solvates, and mixtures thereof.

In one embodiment of the present invention, the pH adjuster is preferably an organic acid or a salt thereof, more preferably an Q-hydroxy acid or a salt thereof, further preferably citric acid or tartaric acid, or a salt thereof, still further preferably anhydrous citric acid or D-tartaric acid, or a salt thereof, particularly preferably anhydrous citric acid.

In one embodiment of the present invention, content of the pH adjuster in the formulation is not particularly limited, but is preferably 0.015 to 5 w/w %, more preferably 0.025 to 1 w/w %, further preferably 0.05 to 0.2 w/w %, especially preferably 0.05 w/w % or higher and lower than 0.1 w/w %, based on the total weight of the formulation.

In one embodiment of the present invention, the pH adjuster is preferably 0.015 to 5 w/w % of anhydrous citric acid, more preferably 0.015 to 1.0 w/w % of anhydrous citric acid, further preferably 0.015 to 0.2 w/w % of anhydrous citric acid, especially preferably 0.015 to 0.075 w/w % of anhydrous citric acid, still more preferably 0.05 to 0.075 w/w % of anhydrous citric acid, based on the total weight of the formulation.

The formulation of the present invention may further contain a non-volatile oil.

In this description, “non-volatile oil” is a pharmaceutically acceptable non-volatile liquid or gel base, which specifically includes non-volatile esters, non-volatile silicones, non-volatile alcohols, non-volatile fatty acids, non-volatile ethers, and the like.

The non-volatile oil is not particularly limited so long as a non-volatile oil that can be used as an additive of pharmaceuticals, can be formulated as a sofpironium bromide formulation in which ingredients are uniformly dispersed or dissolved with ethanol, and does not give an unpleasant feeling of use after application is used.

The non-volatile oil used in the present invention is preferably a non-volatile ester, non-volatile silicone, or non-volatile ether, more preferably a non-volatile ester or non-volatile silicone.

In one embodiment of the present invention, the non-volatile oil is preferably a non-volatile ester.

Non-volatile ester refers to an ester oil that has one or more ester groups (COO⁻) in one molecule, and does not show volatility.

Preferred examples of the non-volatile ester used in the present invention include esters having a linear or branched chain alkyl group having 4 or more carbon atoms.

The non-volatile ester used in the present invention may be any of monoester, diester, and triester.

The term “monoester” refers to an ester having one ester group in one molecule, “diester” refers to an ester having two ester groups in one molecule, and “triester” refers to an ester having three ester groups in one molecule.

When the term “monoester”, “diester”, or “triester” is simply mentioned in this description, they refer to a non-volatile monoester, non-volatile diester, or non-volatile triester, respectively.

In one embodiment of the present invention, the non-volatile oil is a non-volatile ester selected from the group consisting of a monoester, a diester, and a trimester, which is represented as R₁COOR₂, wherein one of R₁ and R₂ is a C₄-C₄₀ straight chain alkyl group that may be substituted, or a C₄-C₄₀ branched chain alkyl group that may be substituted, and the other of R₁ and R₂ is a C₁-C₄₀ alkyl group that may be substituted.

In another embodiment of the present invention, the non-volatile oil is preferably a non-volatile oil selected from the group consisting of a monoester, a diester, and a triester, which is represented as R₁COOR₂, wherein R₁ is a C₄-C₄₀ straight chain alkyl group or C₄-C₄₀ branched chain alkyl group that may be substituted with hydroxyl group or a C₁-C₄ alkyloxycarbonyl group, and R₂ is a C₁-C₄ alkyl group that may be substituted with hydroxyl group or a C₁-C₄₀ alkyloxycarbonyl group.

In another embodiment of the invention, the non-volatile ester is preferably a non-volatile oil selected from the group consisting of a monoester, a diester, and a triester, which is represented as R₁COOR₂, wherein R₁ is a C₄-C₄₀ straight chain alkyl group that may be substituted with a C₁-C₄ alkyloxycarbonyl group, and R₂ is a C₁-C₄ alkyl group that may be substituted with a C₁-C₂₂ alkylcarbonyloxy group.

The “C₄-C₄₀ straight chain alkyl group” means a straight chain alkyl group having 4 to 40 carbon atoms, and refers to normal butyl group, normal pentyl group, normal hexyl group, normal heptyl group, normal octyl group, normal nonyl group, normal undecyl group, normal dodecyl group, normal tridecyl group, normal tetradecyl group, normal pentadecyl group, normal hexadecyl group, normal heptadecyl group, normal octadecyl group, and the like.

The “C₄-C₄₀ branched chain alkyl group” refers to a branched chain alkyl group having from 4 to 40 carbon atoms.

The “C₁-C₄ alkyl group” refers to an alkyl group having 1 to 4 carbon atoms, and may be a straight chain alkyl group or branched chain alkyl group. Specifically, it refers to methyl group, ethyl group, normal propyl group, isopropyl group, normal butyl group, isobutyl group, sec-butyl group, tert-butyl group, and the like.

The “C₁-C₄₀ alkyl group” refers to an alkyl group having 1 to 40 carbon atoms, and may be a C₄-C₄₀ straight chain alkyl group, C₄-C₄₀ branched chain alkyl group, or C₁-C₄ alkyl group.

The expression “may be substituted” used above means that any one or more hydrogen atoms may be replaced with a substituent other than hydrogen. The substituent may be, for example, hydroxyl group, a C₁-C₄ alkyloxycarbonyl group, or a C₄-C₄₀ alkylcarbonyloxy group.

The “C₁-C₄ alkyloxycarbonyl group” means an alkyloxycarbonyl group of which alkyl moiety is the C₁-C₄ alkyl group mentioned above, and it means methyloxycarbonyl group, ethyloxycarbonyl group, normal propyloxycarbonyl group, and the like.

The “C₁-C₄₀ alkylcarbonyloxy group” means an alkylcarbonyloxy group of which alkyl moiety is the C₁-C₄₀ alkyl group mentioned above, and it refers to normal butylcarbonyloxy group, normal hexylcarbonyloxy group, normal heptylcarbonyloxy group, normal octylcarbonyloxy group, and the like.

Examples of the substituents of the “C₄-C₄₀ straight chain alkyl group that may be substituted”, “C₄-C₄₀ branched chain alkyl group that may be substituted”, and “C₁-C₄ alkyl group that may be substituted” include hydroxyl group, a C₁-C₄ alkyloxycarbonyl group, a C₄-C₄₀ alkylcarbonyloxy group, and the like. These groups may be substituted with one or more substituents at any position.

In one embodiment of the present invention, preferred examples of the monoester include, specifically, ethyl myristate, 2-octyldodecyl myristate, butyl stearate, isocetyl stearate, 2-octyldodecyl stearate, hexyl laurate, 2-hexyldecyl laurate, 2-ethylhexyl palmitate, 2-octyldecyl palmitate, cetearyl octanoate, isononyl isononanoate, octyldodecyl neopentanoate, 2-octyldodecyl erucate, 2-octyldodecyl benzoate, a decanoic acid ester, a ricinoleic acid ester, isopropyl myristate, isopropyl palmitate, an alkyl (C₁₄-C₁₈) ethylhexanoate, myristyl myristate, ethyl oleate, oleyl oleate, ethylhexyl palmitate, cetyl palmitate, 2-hexyldecyl myristate, 2-hexyldecyl palmitate, PPG-3 benzyl ether myristate, isotridecyl isononanoate, an alkyl (C₁₂-C₁₅) benzoate, isopropyl isostearate, isostearyl isostearate, PPG-2 myristyl propionate, cetyl 2-ethylhexanoate, 2-ethylhexyl stearate, a medium chain fatty acid monoglyceride, and an alkyl lactate such as ethyl lactate.

The monoester used for the present invention is more preferably isopropyl myristate.

In one embodiment of the present invention, the diester used for the present invention is preferably diisopropyl adipate, di-n-propyl adipate, dioctyl adipate, bis(2-ethylhexyl) adipate, diisostearyl adipate, a medium chain fatty acid diglyceride, or diethyl sebacate.

The diester used in the present invention is more preferably diisopropyl adipate.

In one embodiment of the present invention, preferred examples of the triester used in the present invention are, specifically, triisostearyl citrate, trioctyldodecyl citrate, trioleyl citrate, glyceryl trioctanoate, triethyl citrate, and medium chain fatty acid triglycerides.

The triester used in the present invention is more preferably a medium chain fatty acid triglyceride.

In this description, the medium chain fatty acid triglyceride is a non-volatile component consisting of three molecules of fatty acids ester-bonded to one molecule of glycerol, in which the fatty acids are saturated fatty acids having 6 to 14 carbon atoms. The carbon number of the fatty acids is preferably 8 to 12, and for example, caprylic acid, capric acid, lauric acid, and the like are selected. Preferred medium chain fatty acid triglycerides are caprylic acid triglyceride, capric acid triglyceride, mixtures of caprylic acid and capric acid triglycerides, mixtures of caprylic acid, capric acid and lauric acid triglycerides, tri(caprylic acid/capric acid) glyceride, and the like. For example, Miglyol (registered trademark) 810, 812, and the like can be used.

The medium chain fatty acid triglyceride used in the present invention is preferably tri(caprylic acid/capric acid) glyceride.

In this description, the medium chain fatty acid diglyceride is a non-volatile diester consisting of two molecules of fatty acids ester-bonded to one molecule of glycerol, and the medium chain fatty acid monoglyceride is a non-volatile monoester consisting of one molecule of fatty acid ester-bonded to one molecule of glycerol.

In one embodiment of the present invention, the non-volatile oil may be a non-volatile silicone.

Preferred examples of the non-volatile silicone are, specifically, dimethicone, Silastic (registered trademark), medical grade silicone oil, methyl phenyl silicone, methyl terminated hydrogen branched silicone, decamethylpentacyclosiloxane, octamethyltetracyclosiloxane, dimethylpolysiloxane, methylphenylpolysiloxane, cyclomethicone 5-NF, PEG-12 dimethicone, dimethicone 20 cSt, dimethicone 100 cSt, dimethicone 350 cSt, dimethicone 500 cSt, dimethicone 1000 cSt, dimethicone 12500 cSt, and the like.

More preferred examples of the non-volatile silicone used in the present invention include cyclomethicone 5-NF, PEG-12 dimethicone, dimethicone 20 cSt, and dimethicone 350 cSt.

In one embodiment of the present invention, the formulation may contain two or more kinds of non-volatile oils.

When the formulation of the present invention contains two or more kinds of non-volatile oils, the formulation preferably contains two or more kinds of non-volatile esters selected from the preferred non-volatile esters mentioned above.

For example, the formulation of the present invention may contain two kinds of monoesters, a monoester and a diester, a monoester and a triester, two kinds of diesters, a diester and a triester, two kinds of triesters, a monoester and a non-volatile silicone, a diester and a non-volatile silicone, a triester and a non-volatile silicone, or two kinds of non-volatile silicones.

Content of the non-volatile oil used in the present invention is preferably 0.1 to 50 w/w % based on the total weight of the formulation. The non-volatile oil content is more preferably 0.2 to 25 w/w %, further preferably 0.5 to 10 w/w %, still further preferably 1.0 to 5.0 w/w %, especially preferably 2.5 w/w %, based on the total weight of the formulation.

In one embodiment of the present invention, the non-volatile oil preferably consists of 0.1 to 50 w/w % of isopropyl myristate, diisopropyl adipate, or a medium chain fatty acid triglyceride based on the total weight of the formulation.

In one embodiment of the present invention, the non-volatile oil more preferably consists of 0.5 to 10 w/w % of isopropyl myristate, diisopropyl adipate, or a medium chain fatty acid triglyceride based on the total weight of the formulation.

In one embodiment of the present invention, the non-volatile oil further preferably consists of 1.0 to 5.0 w/w % of isopropyl myristate, diisopropyl adipate, or a medium chain fatty acid triglyceride based on the total weight of the formulation.

In one embodiment of the present invention, the formulation of the present invention may further contain a polyhydric alcohol.

In this description, “polyhydric alcohol” refers to a compound consisting of a hydrocarbon or ether of which plurality of hydrogen atoms at any positions are replaced with hydroxyl groups.

The polyhydric alcohol is not particularly limited so long as a polyhydric alcohol that can be used as an additive in pharmaceuticals is chosen. Examples include hexylene glycol (HG), propylene glycol (PG), ethylene glycol, glycerol, butylene glycol (BG), glycerin, and the like.

In this description, “glycerin” can be concentrated glycerin (cGly), and the like.

In one embodiment of the present invention, the polyhydric alcohol is preferably a compound consisting of a C₂-C₁₀ hydrocarbon of which 2 or 3 hydrogen atoms at any positions are replaced with hydroxyl groups.

In one embodiment of the present invention, the polyhydric alcohol is preferably a compound consisting of a C₂-C₆ hydrocarbon of which 2 or 3 hydrogen atoms at any positions are replaced with hydroxyl groups.

In one embodiment of the present invention, the polyhydric alcohol is preferably hexylene glycol, butylene glycol, or glycerin.

Content of the polyhydric alcohol used in the present invention is preferably 0.1 to 50 w/w % based on the total weight of the formulation. The content of the polyhydric alcohol is more preferably 0.5 to 40 w/w %, further preferably 1.0 to 30 w/w %, still further preferably 2.0 to 20/w %, especially preferably 10 w/w %, based on the total weight of the formulation.

In one embodiment of the present invention, the polyhydric alcohol preferably consists of 0.1 to 50 w/w % of hexylene glycol, butylene glycol, or glycerin based on the total weight of the formulation.

In one embodiment of the present invention, the polyhydric alcohol more preferably consists of 0.1 to 30 w/w % of hexylene glycol, butylene glycol, or glycerin based on the total weight of the formulation.

In one embodiment of the present invention, the polyhydric alcohol further preferably consists of 2.0 to 20 w/w % of hexylene glycol, butylene glycol, or glycerin based on the total weight of the formulation.

The “viscosity” used in this description is synonymous with consistency, and indicates the degree of resistance to flow of a liquid.

The term “viscosity of the formulation” used in this description means the viscosity at 25° C. unless especially noted. Viscosity can generally be easily measured by the method described in the Japanese Pharmacopoeia, General Test Methods, as well as other test methods commonly used by those skilled in the art. For example, it can be measured by the viscosity measurement method shown in the following examples.

In one embodiment of the present invention, the viscosity of the formulation of the present invention is preferably 10 to 2000 mPa·s, more preferably 100 to 1500 mPa·s, at 25° C.

In another embodiment of the present invention, the viscosity of the formulation of the present invention is preferably 10 to 1000 mPa·s, more preferably 100 to 800 mPa·s, at 25° C.

In another embodiment of the present invention, the viscosity of the formulation of the present invention is preferably 10 to 800 mPa·s, more preferably 50 to 800 mPa·s, at 25° C.

In this description, the expression “at the time of preparation of the formulation” refers to the time when or immediately after the formulation is prepared. Unless especially noted, it is synonymous with the time at which the product is manufactured. Typically, it refers to a time point within a period of one week after the preparation of the formulation, preferably 5 days after the preparation, more preferably 3 days after the preparation.

In this description, the expression “decrease in viscosity over time” refers broadly to the phenomenon of decrease by 30% or more in the viscosity of the formulation after the formulation is stored for an arbitrary period of time relative to the viscosity at the time of the preparation of the formulation, and more narrowly to the phenomenon of the decrease by 20% or more, 10% or more, or 5% or more.

In one embodiment of the present invention, the decrease in viscosity over time mentioned in this description refers to a phenomenon that the value of the viscosity observed after storage for 6 months at room temperature, 12 months at room temperature, 24 months at room temperature, or 36 months at room temperature is lower than the value of the viscosity at the time of the preparation of the formulation. Unless especially stated, viscosity refers to the value of viscosity measured at 25° C.

In one embodiment of the present invention, the decrease in viscosity over time mentioned in this description refers to a phenomenon that the value of the viscosity observed after storage for 1 month at 40° C., 2 months at 40° C., or 3 months at 40° C. is lower than the value at the time of the preparation of the formulation. Unless especially stated, viscosity refers to the value of viscosity measured at 25° C.

In one embodiment of the present invention, the viscosity increase or decrease rate of the formulation of the present invention after it is stored at room temperature for 12 months from the preparation is within ±30%, preferably within ±20%, more preferably within ±10%, further preferably within ±5%, relative to the viscosity at the time of the preparation of the formulation.

In one embodiment of the present invention, the viscosity increase or decrease rate of the formulation of the present invention after it is stored at room temperature for 24 months from the preparation is within ±30%, preferably within ±20%, more preferably within ±10%, further preferably within ±5%, relative to the viscosity at the time of the preparation of the formulation.

In one embodiment of the present invention, the viscosity increase or decrease rate of the formulation of the present invention after it is stored at room temperature for 36 months from the preparation is within ±30%, preferably within ±20%, more preferably within ±10%, further preferably within ±5%, relative to the viscosity at the time of the preparation of the formulation.

In one embodiment of the present invention, the viscosity increase or decrease rate of the formulation of the present invention after it is stored at 40° C. for 3 months from the preparation is within ±30%, preferably within ±20%, more preferably within ±10%, further preferably within ±5%, relative to the viscosity at the time of the preparation of the formulation.

The formulation of the present invention is stable for a long period of time, and produces almost no decomposition products during the storage period, and therefore it is suitable for a pharmaceutical product.

In one embodiment of the present invention, content of the compound (II) represented by the following formula (II)

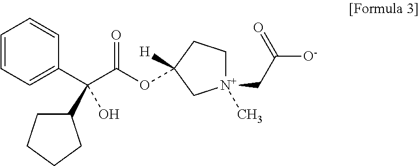

and epimer at N⁺
in the formulation of the present invention is 1.5 w/w % or lower based on the content of sofpironium bromide, and purity of sofpironium bromide in the same is 90 w/w % or higher, after storage at room temperature for 24 months from the preparation of the formulation, after storage at room temperature for 36 months from the preparation of the formulation, or after storage at 40° C. for 3 months from the preparation of the formulation.

In another embodiment of the present invention, the content of the compound (II) in the formulation of the present invention is 1.5 w/w % or lower based on the content of sofpironium bromide, and purity of sofpironium bromide in the same is 95 w/w % or higher, after storage at room temperature for 24 months from the preparation of the formulation, after storage at room temperature for 36 months from the preparation of the formulation, or after storage at 40° C. for 3 months from the preparation of the formulation.

In another embodiment of the present invention, the content of the compound (II) in the formulation of the present invention is 1.5 w/w % or lower based on the content of sofpironium bromide, and purity of sofpironium bromide in the same is 98 w/w % or higher, after storage at room temperature for 24 months from the preparation of the formulation, after storage at room temperature for 36 months from the preparation of the formulation, or after storage at 40° C. for 3 months from the preparation of the formulation.

In another embodiment of the present invention, the content of the compound (II) in the formulation of the present invention is 0.4 w/w % or lower based on the content of sofpironium bromide, and purity of sofpironium bromide in the same is 99.6 w/w % or higher, after storage at room temperature for 24 months from the preparation of the formulation, after storage at room temperature for 36 months from the preparation of the formulation, or after storage at 40° C. for 3 months from the preparation of the formulation.

In another embodiment of the present invention, the content of the compound (II) in the formulation of the present invention is 1.5 w/w % or lower based on the content of sofpironium bromide, total content of impurities other than the compound (II) in the same is 1.0 w/w % or lower based on the content of sofpironium bromide, and purity of sofpironium bromide in the same is 98 w/w % or higher, after storage at room temperature for 24 months from the preparation of the formulation, after storage at room temperature for 36 months from the preparation of the formulation, or after storage at 40° C. for 3 months from the preparation of the formulation.

In another embodiment of the present invention, the content of the compound (II) in the formulation of the present invention is 1.5 w/w % or lower based on the content of sofpironium bromide, total content of impurities other than the compound (II) in the same is 0.5 w/w % or lower based on the content of sofpironium bromide, and purity of sofpironium bromide in the same is 98 w/w % or higher, after storage at room temperature for 24 months from the preparation of the formulation, after storage at room temperature for 36 months from the preparation of the formulation, or after storage at 40° C. for 3 months from the preparation of the formulation.

In another embodiment of the present invention, the content of the compound (II) in the formulation of the present invention is 0.4 w/w % or lower based on the content of sofpironium bromide, total content of impurities other than the compound (II) in the same is 0.4 w/w % or lower based on the content of sofpironium bromide, and purity of sofpironium bromide in the same is 99.6 w/w % or higher, after storage at room temperature for 24 months from the preparation of the formulation, after storage at room temperature for 36 months from the preparation of the formulation, or after storage at 40° C. for 3 months from the preparation of the formulation.

Hereafter, uses of the formulation of the present invention and typical examples of the uses are explained.

The formulation of the present invention can be used for medical care, therapeutic treatment, or prevention of diseases for which efficacy based on the anticholinergic action of the active ingredient, sofpironium bromide, can be expected, especially primary hyperhidrosis, overactive bladder, chronic obstructive pulmonary disease, cardiac disease, salivation, eye disease, bronchial asthma, and the like.

The formulation of the present invention can be preferably used for the therapeutic treatment or prevention of hyperhidrosis, more preferably for the therapeutic treatment or prevention of focal hyperhidrosis.

In one embodiment of the present invention, the formulation of the present invention can be used for medical care, therapeutic treatment, or prevention of primary axillary hyperhidrosis.

In another embodiment of the present invention, the formulation of the present invention can be used for medical care, therapeutic treatment, or prevention of primary palmar hyperhidrosis.

In one embodiment of the present invention, the formulation of the present invention for external application is used for medical care, therapeutic treatment, or prevention of, in particular, primary axillary hyperhidrosis, and a pharmaceutically acceptable formulation containing 1 to 15 w/w % of sofpironium bromide, preferably 5 w/w % of sofpironium bromide, is topically administered to the axilla, preferably to the both axillae, once a day for a treatment period of at least 6 weeks.

### EXAMPLES

Hereafter, the formulation of the present invention is explained in more detail with reference to the test examples as examples. However, it is not intended to limit the present invention to these examples.

### Test Example 1

### Accelerated Test of Sofpironium Bromide in Various Non-Aqueous Formulations (1)

### <Formulation Preparation Method>

The ingredients shown in the following table were stirred and dissolved in anhydrous ethanol at the concentrations thereof shown in the table to obtain formulations. The compositions of the formulations prepared in this example are shown in the table mentioned below.

TABLE 1

| | Ingredient and concentration (w/w %) | | | | | |
|---|---|---|---|---|---|---|
| | | | | | Water- | |
| | Active | Non-volatile | Polyhydric | | soluble | |
| Formulation | ingredient | ester | alcohol | pH adjuster | polymer | Solvent |
| Example 1 | BBI-4000 | IPM* | HG* | Anhydrous citric | HPC | Anhydrous |
| | (5) | (2.5) | (10) | acid | (1.25) | ethanol |
| | | | | (0.05) | | (q.s. to 100**) |
| Example 2 | BBI-4000 | IPM | None | Anhydrous citric | HPC | Anhydrous |
| | (5) | (2.5) | | acid | (1.25) | ethanol |
| | | | | (0.05) | | (q.s. to 100) |
| Example 3 | BBI-4000 | DIA* | HG | Anhydrous citric | HPC | Anhydrous |
| | (5) | (2.5) | (10) | acid | (1.25) | ethanol |
| | | | | (0.05) | | (q.s. to 100) |
| Example 4 | BBI-4000 | MCT* | HG | Anhydrous citric | HPC | Anhydrous |
| | (5) | (2.5) | (10) | acid | (1.25) | ethanol |
| | | | | (0.05) | | (q.s. to 100) |
| Example 5 | BBI-4000 | IPM | BG* | Anhydrous citric | HPC | Anhydrous |
| | (5) | (2.5) | (10) | acid | (1.25) | ethanol |
| | | | | (0.05) | | (q.s. to 100) |
| Example 6 | BBI-4000 | IPM | cGly* | Anhydrous citric | HPC | Anhydrous |
| | (5) | (2.5) | (10) | acid | (1.25) | ethanol |
| | | | | (0.05) | | (q.s. to 100) |
| Example 7 | BBI-4000 | IPM | HG | D-TA | HPC | Anhydrous |
| | (5) | (2.5) | (10) | (0.05) | (1.25) | ethanol |
| | | | | | | (q.s. to 100) |
| Comparative | BBI-4000 | IPM | BG | Anhydrous citric | HPC | Anhydrous |
| Example 1 | (5) | (2.5) | (10) | acid (0.001) | (1.25) | ethanol |
| | | | | | | (q.s. to 100) |
| Comparative | BBI-4000 | IPM | HG | Acetic acid | HPC | Anhydrous |
| Example 2 | (5) | (2.5) | (10) | (0.05) | (1.25) | ethanol |
| | | | | | | (q.s. to 100) |
| Comparative | BBI-4000 | IPM | HG | 1 mol/L | HPC | Anhydrous |
| Example 3 | (5) | (2.5) | (10) | Hydrochloric acid | (1.25) | ethanol |
| | | | | (0.05) | | (q.s. to 100) |

*IPM: isopropyl myristate, HG^{:} hexylene glycol, D-TA: D-tartaric acid, DIA: diisopropyl adipate, MCT medium chain fatty acid triglyceride, BG: butylene glycol, cGly: concentrated glycerin
**q.s. to 100: The remainder is constituted with anhydrous ethanol so that the total amount should be 100%.

### <Viscosity Measurement Method>

In a viscometer set at 25° C., 5 rpm, and preheat time of 30 seconds, approximately 1 mL of each formulation was rotated for 200 seconds in a cone rotor: R—H1° 34′×R24, and then viscosity was measured (Japanese Pharmacopoeia, Viscosity measurement method 2).

TABLE 2

| Type | Model | Manufacturer |
|---|---|---|
| E-type viscometer | RE550H | Toki Sangyo Co., Ltd. |

### <pH Measurement Method>

In order to suppress the fluctuation of pH value, the amount of the test formulation must be within a certain range relative to the internal liquid from the pH electrode. In the pH measurement of this test example, the amount of the test product was 10.0 g.

Oxalate pH standard solutions, phthalate pH standard solutions, or phosphate pH standard solutions were used to calibrate the pH electrode. The temperature difference between the pH standard solutions used for calibration and the test formulation was ±2° C. The temperature of the formulation at the time of pH measurement was in the range of 20 to 30° C.

The test formulation was weighed in an amount of 10.0 g in Maruemu (registered trademark) No. 4 bottle, and then the pH value was measured with a pH electrode for low conductivity water/non-aqueous solvent calibrated by using the pH standard solutions after the electrode was immersed in the formulation for 5 minutes.

TABLE 3

| | Model or | |
|---|---|---|
| Type | specification | Manufacturer |
| pH electrode for low conductivity | 6377-10D | Horiba, Ltd. |
| water/non-aqueous solvent | | |
| pH meter | F-52 | Horiba, Ltd. |

### <Stability Test Method: Storage Under 40±2° C./75±5% RH and Light Shielding for 3-Months>

The viscosity increase/decrease rates of the formulations after storage at 40° C. for 3 months from the preparation of the formulation relative to the viscosity at the time of the preparation were calculated, results within ±30% were determined as “A”, and those exceeding ±30% were determined as “B”. The results are shown in the table mentioned below.

The pH values shown in the table mentioned below were the highest values during the storage period (i.e., up to the third month after the preparation of the formulation).

TABLE 4

| | | Viscosity (mPa · s) | | | Increase/decrease | |
|---|---|---|---|---|---|---|
| | | At the time of | After 1 | After 3 | rate of viscosity after | |
| Formulation | pH | preparation | month | months | 3 months (%) | Judgment |
| Example 1 | 3.8 | 591 | 575 | 607 | +2.7% | A |
| Example 2 | 3.9 | 529 | 517 | 543 | +2.6% | A |
| Example 3 | 3.7 | 631 | 625 | 634 | +0.48% | A |
| Example 4 | 3.6 | 634 | 643 | 640 | +0.95% | A |
| Example 5 | 3.8 | 659 | 663 | 675 | +2.4% | A |
| Example 6 | 3.7 | 691 | 712 | 717 | +3.8% | A |
| Example 7 | 3.9 | 597 | 607 | 605 | +1.3% | A |
| Comparative | 6.0 | 639 | 655 | 381 | −40% | B |
| Example 1 | | | | | | |
| Comparative | 5.4 | 607 | 591 | 362 | −40% | B |
| Example 2 | | | | | | |
| Comparative | 6.9 | 620 | 654 | 164 | −74% | B |
| Example 3 | | | | | | |

TABLE 5

| | Ingredient and | pH | | |
|---|---|---|---|---|
| | concentration | At the | | |
| | (w/w %) | time of | After 1 | After 3 |
| Formulation | pH adjuster | preparation | month | months |
| Example 1 | Anhydrous citric | 3.6 | 3.6 | 3.8 |
| | acid (0.05) | | | |
| Example 2 | Anhydrous citric | 3.6 | 3.6 | 3.9 |
| | acid (0.05) | | | |
| Example 3 | Anhydrous citric | 3.6 | 3.6 | 3.7 |
| | acid (0.05) | | | |
| Example 4 | Anhydrous citric | 3.5 | 3.5 | 3.6 |
| | acid (0.05) | | | |
| Example 5 | Anhydrous citric | 3.6 | 3.6 | 3.8 |
| | acid (0.05) | | | |
| Example 6 | Anhydrous citric | 3.6 | 3.6 | 3.7 |
| | acid (0.05) | | | |
| Example 7 | D-TA | 3.7 | 3.7 | 3.9 |
| | (0.05) | | | |
| Comparative | Anhydrous citric | 6.0 | 5.1 | 4.6 |
| Example 1 | acid (0.001) | | | |
| Comparative | Acetic acid | 5.4 | 5.2 | 5.2 |
| Example 2 | (0.05) | | | |
| Comparative | 1 mol/L Hydrochloric | 6.9 | 5.1 | 4.7 |
| Example 3 | acid (0.05) | | | |

Surprisingly, the pH values of the sofpironium bromide compositions varied over time, and when the highest pH value was 5.4 or higher, the viscosity imparted by the water-soluble polymer decreased over time (Comparative Examples 1 to 3).

On the other hand, as shown by Examples 1 to 7, it was revealed that when the pH values of the sofpironium bromide formulations are maintained at 5.2 or lower, the decrease in viscosity is suppressed.

### Test Example 2

### Accelerated Test of Sofpironium Bromide in Various Non-Aqueous Formulations (2)

### <Formulation Preparation Method>

Compositions of Reference Examples 1 to 5 (formulations not containing sofpironium bromide), Comparative Example 4, and Examples 8 to 12 were prepared in the same manner as in Test Example 1. These formulations were used for viscosity stability test.

The formulations of Reference Examples 1 to 5, Comparative Example 4, and Examples 8 to 12 were formulations containing sofpironium bromide, anhydrous citric acid, anhydrous ethanol, IPM (2.5 w/w %), HG (10 w/w %), and HPC (1.25 w/w %). The contents of sofpironium bromide and anhydrous citric acid are shown in Table 7. The remainder was constituted with anhydrous ethanol so that the total amount should be 100%.

### <Test Method>

The pH measurement method was the same as in Test Example 1.

### <Viscosity Measurement Method>

For the formulations containing 0% and 5% BBI-4000, viscosity was measured with a viscometer set at 25° C., 10 rpm, and preheating time of 30 seconds, after approximately 1 mL of the sample was rotated for 200 seconds in a cone rotor: R—H1° 34′×R24 (viscosity measurement method 2). For the formulations containing 15% BBI-4000, viscosity was measured with a viscometer set at 25° C., 7 rpm, and preheating time of 30 seconds, after approximately 1 mL of the sample was rotated for 200 seconds in a cone rotor: R—H1° 34′×R24 (Japanese Pharmacopoeia viscosity measurement method 2).

TABLE 6

| Type | Model | Manufacturer |
|---|---|---|
| E-type viscometer | RE550H | Toki Sangyo Co., Ltd. |

<Stability Test Method: Storage for 3 Months Under 40±2° C./75±5% RH with Light Shielding>

In the tables mentioned below, the definitions of pH, Measurement method, and Judgment criteria were the same as in Test Example 1.

TABLE 7

| | Ingredient and | | | | | Viscosity | |
|---|---|---|---|---|---|---|---|
| | concentration (w/w %) | | | Viscosity (mPa · s) | | increase/decrease | |
| | BBI- | Anhydrous | | At the time of | After 3 | rate after 3 | |
| Formulation | 4000 | citric acid | pH | preparation | months | months (%) | Judgment |
| Reference | 0 | 0.001 | 7.0 | 330 | 378 | +15 | A |
| Example 1 | | | | | | | |
| Reference | | 0.015 | 5.2 | 326 | 336 | +3.1 | A |
| Example 2 | | | | | | | |
| Reference | | 0.025 | 4.9 | 329 | 361 | +9.7 | A |
| Example 3 | | | | | | | |
| Reference | | 0.050 | 4.5 | 329 | 378 | +15 | A |
| Example 4 | | | | | | | |
| Reference | | 0.075 | 4.4 | 332 | 343 | +3.3 | A |
| Example 5 | | | | | | | |
| Comparative | 5 | 0.001 | 5.5 | 363 | 76.4 | −79 | B |
| Example 4 | | | | | | | |
| Example 8 | 5 | 0.015 | 5.2 | 347 | 313 | −9.8 | A |
| Example 9 | | 0.025 | 4.8 | 364 | 351 | −3.6 | A |
| Example 10 | | 0.050 | 4.4 | 369 | 358 | −2.9 | A |
| Example 11 | | 0.075 | 4.1 | 367 | 374 | +1.9 | A |
| Example 12 | 15 | 0.050 | 3.9 | 737 | 702 | −4.7 | A |

TABLE 8

| | Ingredient concentration | | | | | |
|---|---|---|---|---|---|---|
| | (w/w %) | | pH | | | |
| | | Anhydrous | At the time of | After 1 | After 2 | After 3 |
| Formulation | BBI-4000 | citric acid | preparation | month | months | months |
| Reference | 0 | 0.001 | 6.5 | 6.9 | 6.7 | 7.0 |
| Example 1 | | | | | | |
| Reference | | 0.015 | 5.0 | 5.2 | 5.1 | 5.2 |
| Example 2 | | | | | | |
| Reference | | 0.025 | 4.4 | 4.9 | 4.8 | 4.9 |
| Example 3 | | | | | | |
| Reference | | 0.050 | 4.2 | 4.5 | 4.5 | 4.5 |
| Example 4 | | | | | | |
| Reference | | 0.075 | 4.2 | 4.4 | 4.4 | 4.4 |
| Example 5 | | | | | | |
| Comparative | 5 | 0.001 | 5.5 | 5.1 | 4.5 | 4.6 |
| Example 4 | | | | | | |
| Example 8 | 5 | 0.015 | 4.0 | 4.0 | 4.7 | 5.2 |
| Example 9 | | 0.025 | 3.9 | 4.0 | 4.5 | 4.8 |
| Example 10 | | 0.050 | 3.7 | 3.8 | 4.1 | 4.4 |
| Example 11 | | 0.075 | 3.5 | 3.7 | 3.9 | 4.1 |
| Example 12 | 15 | 0.050 | 3.4 | 3.6 | 3.7 | 3.9 |

When sofpironium bromide was not contained, the viscosity increase or decrease rate was slight, even when the pH value became significantly high (Reference Example 1 and the like), and no relationship was observed between pH and viscosity increase or decrease rate (Reference Examples 1 to 5). These results indicate that the decrease in viscosity over time in non-aqueous formulations of sofpironium bromide is a very special phenomenon that becomes apparent only when sofpironium bromide is contained in the formulation.

When the highest pH value was 5.5 up to the third month after the preparation of the formulation, the decrease in viscosity over time was significant (Comparative Example 4). On the other hand, when the pH was maintained at 5.2 or lower for a period of 3 months after the preparation of the formulation, the decrease in viscosity over time was slight or not observed (Examples 8 to 12). This tendency was also true when the concentration of sofpironium bromide was 15 w/w % (Example 12). After the formulation of Example 10 was stored at 40° C. for 6 months, the viscosity thereof was 322 mPa·s. The viscosity increase or decrease rate relative to that at the time of the preparation of the formulation was −13%, indicating that the stability was maintained even after 6 months.

### <Purity Test>

The results of purity test (related substances) of the formulation of Example 10 (5% BBI-4000 gel formulation (citric acid concentration, 0.050%)) conducted in the stability test for 3 months at 40±2° C./75±5% RH with light shielding are shown in the table mentioned below.

TABLE 9

| | HPLC area percentage (%) | | | |
|---|---|---|---|---|
| | At the time of | After 1 | After 2 | After 3 |
| Relative retention time | preparation | month | months | months |
| 0.60~0.66* | — | — | <0.04** | <0.04** |
| 0.68* | — | — | — | — |
| 1.70~1.72*** | — | — | <0.04** | <0.04** |

*: Compound (II) for all the cases
**Lower than quantification limit (0.04%)
***: Ethyl cyclopentylmandelate
—: Not detected

The compound (II) is a compound generated by hydrolysis of ethyl ester of sofpironium bromide, and is represented by the following formula.

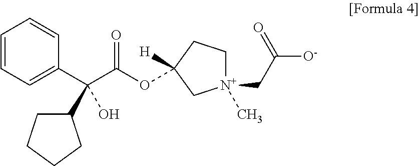

and epimer at N⁺

In the above purity test, the only analogues detected in an amount exceeding 0.1% were the compound (II) and ethyl cyclopentylmandelate. Therefore, it was demonstrated that the non-aqueous formulation of sofpironium bromide of the present invention is an extremely stable composition in which almost no analogues (including impurities) are generated during storage period.

### Test Example 3

### Accelerated Test of Sofpironium Bromide in Various Low Water Content Formulations

### <Formulation Preparation Method>

The compositions of Examples 13 to 15 were prepared in the same manner as in Test Example 1. The ingredients were stirred and dissolved in anhydrous ethanol to obtain formulations so that the formulations should contain the ingredients shown in the table at the concentrations shown in the table. The compositions of the formulations prepared in this example are shown in the table mentioned below.

TABLE 10

| | Ingredient and concentration (w/w %) | | | | | | |
|---|---|---|---|---|---|---|---|
| | | Non- | | | Water- | | |
| | Active | volatile | Polyhydric | | soluble | | |
| Formulation | ingredient | ester | alcohol | pH adjuster | polymer | Water | Solvent |
| Example 13 | BBI-4000 | IPM | HG | Anhydrous citric | HPC | Water | Anhydrous |
| | (5) | (2.5) | (10) | acid | (1.25) | (1) | ethanol |
| | | | | (0.05) | | | (q.s. to 100) |
| Example 14 | BBI-4000 | IPM | HG | Anhydrous citric | HPC | Water | Anhydrous |
| | (5) | (2.5) | (10) | acid | (1.25) | (2) | ethanol |
| | | | | (0.05) | | | (q.s. to 100) |
| Example 15 | BBI-4000 | IPM | HG | Anhydrous citric | HPC | Water | Anhydrous |
| | (5) | (2.5) | (10) | acid | (1.25) | (5) | ethanol |
| | | | | (0.05) | | | (q.s. to 100) |

** q.s. to 100: The reminc er was constituted with anhydrous ethanol so that the total amount should be 100%.

### <Test Method>

The pH measurement method was the same as in Test Example 1, and the viscosity measurement method was the same as in Test Example 2.

<Stability Test Method: Storage for 3 Months Under 40±2° C./75±5% RH with Light Shielding>

In the tables mentioned below, the definitions of pH and the judgment criteria were the same as in Test Example 1.

TABLE 11

| | | Viscosity (mPa · s) | | Viscosity | |
|---|---|---|---|---|---|
| | | At the | | increase/decrease | |
| | | time of | After 3 | rate after 3 | |
| Formulation | pH | preparation | months | months (%) | Judgment |
| Example 13 | 3.7 | 454 | 442 | −2.6 | A |
| Example 14 | 3.7 | 484 | 459 | −5.2 | A |
| Example 15 | 3.7 | 534 | 512 | −4.1 | A |

TABLE 12

| | Ingredient and | | | | |
|---|---|---|---|---|---|
| | concentration | pH | | | |
| | (w/w %) | At the | | | |
| | Anhydrous | time of | After 1 | After 2 | After 3 |
| Formulation | citric acid | preparation | month | months | months |
| Example 13 | 0.05 | 3.5 | 3.6 | 3.7 | 3.7 |
| Example 14 | 0.05 | 3.6 | 3.6 | 3.7 | 3.7 |
| Example 15 | 0.05 | 3.7 | 3.7 | 3.7 | 3.5 |

As indicated in the table mentioned above, the viscosity increase or decrease rates observed in Examples 13 to 15 using a water content of 5 w/w % or lower were slight. In other words, it was revealed that, as in the case of the non-aqueous formulations of sofpironium bromide, the viscosity decrease over time was slight also in low water content formulations with a water content of 5 w/w % or lower by maintaining the pH value at 5.2 or lower.

### <Purity Test>

The results of purity test (related substances) of the formulations of Examples 13 to 15 (5% BBI-4000 gel formulations (citric acid concentration, 0.050%)) conducted in the stability test for 3 months at 40±2° C./75±5% RH with light shielding are shown in the table mentioned below.

TABLE 13

| | | HPLC area percentage (%) | | | |
|---|---|---|---|---|---|
| | Relative | At the | | | |
| | retention | time of | After 1 | After 2 | After 3 |
| Formulation | time | preparation | month | months | months |
| Example 13 | 0.60~0.61* | — | — | — | 0.05 |
| | 0.68* | — | — | — | — |
| | 1.70*** | — | — | — | — |
| Example 14 | 0.60~0.63* | — | — | 0.05 | 0.09 |
| | 0.68~0.70* | — | — | <0.04** | 0.05 |
| | 1.70*** | — | — | — | — |
| Example 15 | 0.60~0.63* | — | 0.07 | 0.14 | 0.21 |
| | 0.68~0.70* | — | <0.04** | 0.09 | 0.13 |

*Compound (II) for all the cases.
**Lower than quantification limit (0.04%)
***Ethyl cyclopentylmandelate
— Not detected

On the basis of the results shown in the table mentioned above, it was confirmed that although the amount of the compound (II) formed by hydrolysis slightly increased with increase of the amount of water added, other related substances were hardly generated.

As described above, it was demonstrated that a low water content formulation of sofpironium bromide is a stable formulation in which related substances are generated in extremely small amounts, at least when the water content is 5 w/w % or lower.

On the basis of the results described above, it was revealed that a low water content formulation of sofpironium bromide with a water content of 5 w/w % or lower shows almost no decrease in viscosity over time, and limited formation of analogous substances, and thus it shows a good profile as a pharmaceutical formulation, like the non-aqueous formulation.

### Test Example 4

### Accelerated Test of Sofpironium Bromide in Various Non-Aqueous Formulations (3)

### <Formulation Preparation Method>

The formulations of Examples 16 to 19 were prepared in the same manner as in Test Example 1. The ingredients were stirred and dissolved in anhydrous ethanol to obtain formulations so that the formulations should contain the ingredients shown in the table at the concentrations shown in the table. The compositions of the formulations prepared in this example are shown in the table mentioned below.

TABLE 14

| | Ingredient and concentration (w/w %) | | | | | |
|---|---|---|---|---|---|---|
| | | Non- | | | Water- | |
| | Active | volatile | Polyhydric | | soluble | |
| Formulation | ingredient | ester | alcohol | pH adjuster | polymer | Water |
| Example 16 | BBI-4000 | None | None | Anhydrous | HPC | Anhydrous |
| | (5) | | | citric acid | (1.25) | ethanol |
| | | | | (0.05) | | (q.s. to 100)** |
| Example 17 | BBI-4000 | None | None | Acetic acid | HPC | Anhydrous |
| | (5) | | | (1.2) | (1.25) | ethanol |
| | | | | | | (q.s. to 100) |
| Example 18 | BBI-4000 | None | None | 1 mol/L | HPC | Anhydrous |
| | (5) | | | hydrochloric | (1.25) | ethanol |
| | | | | acid | | (q.s. to 100) |
| | | | | (0.3) | | |
| Example 19 | BBI-4000 | None | None | D-TA* | HPC | Anhydrous |
| | (5) | | | (0.05) | (1.25) | ethanol |
| | | | | | | (q.s. to 100) |

*D-TA: D-Tartaric acid
**q.s. to 100: The reminder was constituted with anhydrous ethanol so that the total amount should be 100%.

### <Test Method>

The pH measurement method and viscosity measurement method were the same as in Test Example 1.

<Stability Test Method: Storage for 3 Months Under 40±2° C./75±5% RH with Light Shielding>

In the tables mentioned below, the definitions of pH and the judgment criteria were the same as in Test Example 1.

TABLE 15

| | | | | | Viscosity | |
|---|---|---|---|---|---|---|
| | | Viscosity (mPa · s) | | | increase/ | |
| | | At the | | | decrease | |
| | | time of | After 1 | After 3 | rate after 3 | |
| Formulation | pH | preparation | month | months | months (%) | Judgment |
| Example 16 | 3.7 | 501 | 493 | 448 | −10.6 | A |
| Example 17 | 4.0 | 521 | 501 | 561 | +7.7 | A |
| Example 18 | 4.2 | 522 | 508 | 506 | −3.1 | A |
| Example 19 | 3.7 | 490 | 489 | 553 | +12.9 | A |

TABLE 16

| | Ingredient and | pH | | |
|---|---|---|---|---|
| | concentration | At the | | |
| | (w/w %) | time of | After 1 | After 3 |
| Formulation | pH adjuster | preparation | month | months |
| Example 16 | Anhydrous citric | 3.5 | 3.6 | 3.7 |
| | acid (0.05) | | | |
| Example 17 | Acetic acid | 4.0 | 4.0 | 4.0 |
| | (1.2) | | | |
| Example 18 | 1 mol/L hydrochloric | 4.2 | 4.2 | 4.1 |
| | acid (0.3) | | | |
| Example 19 | D-TA | 3.6 | 3.6 | 3.7 |
| | (0.05) | | | |

As shown by Examples 16 to 19, it was revealed that viscosity decrease is suppressed also in non-aqueous sofpironium bromide formulations not containing non-volatile oil and polyhydric alcohol, if the pH values of the formulations are maintained at 5.2 or lower.

### Test Example 5

### Accelerated Test of Sofpironium Bromide in Various Non-Aqueous Formulations (4)

### <Formulation Preparation Method>

The formulations of Examples 20 to 23 were prepared in the same manner as in Test Example 1. The ingredients were stirred and dissolved in anhydrous ethanol to obtain formulations so that the formulations should contain the ingredients shown in the table at the concentrations shown in the table. The compositions of the formulations prepared in this example are shown in the table mentioned below.

TABLE 17

| | Ingredient and concentration (w/w %) | | | | | |
|---|---|---|---|---|---|---|
| | | Non- | | | Water- | |
| | Active | volatile | Polyhydric | | soluble | |
| Formulation | ingredient | ester | alcohol | pH adjuster | polymer | Solvent |
| Example 20 | BBI-4000 | IPM* | HG* | Acetic acid | HPC | Anhydrous |
| | (5) | (2.5) | (10) | (1.2) | (1.25) | ethanol |
| | | | | | | (q.s. to 100)** |
| Example 21 | BBI-4000 | CyM* | HG | Anhydrous | HPC | Anhydrous |
| | (5) | (2.5) | (10) | citric acid | (1.25) | ethanol |
| | | | | (0.05) | | (q.s. to 100) |
| Example 22 | BBI-4000 | PD* | HG | Anhydrous | HPC | Anhydrous |
| | (5) | (2.5) | (10) | citric acid | (1.25) | ethanol |
| | | | | (0.05) | | (q.s. to 100) |
| Example 23 | BBI-4000 | IPM | HG | Anhydrous | CVP* | Anhydrous |
| | (5) | (2.5) | (10) | citric acid | (1.25) | ethanol |
| | | | | (0.05) | | (q.s. to 100) |

*IPM: Isopropyl myristate, HG: Hexylene glycol, CVP: Carboxyvinyl polymer, CyM Cyclomethicone, PD: PEG-12 dimethicone
**q.s. to 100: The reminder was constituted with anhydrous ethanol so that the total amount should be 100%.

### <Test Method>

The pH measurement method and viscosity measurement method were the same as in Test Example 1.

<Stability Test Method: Storage for 3 Months Under 40±2° C./75±5% RH with Light Shielding>

In the tables mentioned below, the definitions of pH and the judgment criteria were the same as in Test Example 1.

TABLE 18

| | | | | | Viscosity | |
|---|---|---|---|---|---|---|
| | | Viscosity (mPa · s) | | | increase/ | |
| | | At the | | | decrease | |
| | | time of | After 1 | After 3 | rate after 3 | |
| Formulation | pH | preparation | month | months | months (%) | Judgment |
| Example 20 | 4.3 | 591 | 603 | 557 | −5.8 | A |
| Example 21 | 3.9 | 573 | 575 | 557 | −2.8 | A |
| Example 22 | 3.8 | 612 | 603 | 594 | −2.9 | A |
| Example 23 | 3.0 | 617 | 594 | 571 | −7.5 | A |

TABLE 19

| | Ingredient and | pH | | |
|---|---|---|---|---|
| | concentration | At the | | |
| | (w/w %) | time of | After 1 | After 3 |
| Formulation | pH adjuster | preparation | month | months |
| Example 20 | Acetic acid | 3.9 | 4.0 | 4.3 |
| | (1.2) | | | |
| Example 21 | Anhydrous citric | 3.5 | 3.6 | 3.9 |
| | acid (0.05) | | | |
| Example 22 | Anhydrous citric | 3.6 | 3.7 | 3.8 |
| | acid (0.05) | | | |
| Example 23 | Anhydrous citric | 3.0 | 2.8 | 2.7 |
| | acid (0.05) | | | |

As shown by Examples 20 to 23, it was revealed that, regardless of the type of non-volatile oil or water-soluble polymer used, if the pH value of the sofpironium bromide formulation was maintained at 5.2 or lower, viscosity decrease was suppressed.

### Test Example 6

### Long-Term Storage Test of Sofpironium Bromide Formulations

### <Formulation Preparation Method>

The formulations of Examples 24, 25, Comparative Examples 5 and 6 were prepared in the same manner as in Test Example 1, and used for various tests. The ingredients were stirred and dissolved in anhydrous ethanol to obtain formulations so that the formulations should contain the ingredients shown in the table at the concentrations shown in the table. The compositions of the formulations prepared in this example are shown in the table mentioned below.

TABLE 20

| | Ingredient and concentration (w/w %) | | | | | |
|---|---|---|---|---|---|---|
| | | Non- | | | Water- | |
| | Active | volatile | Polyhydric | | soluble | |
| Formulation | ingredient | ester | alcohol | pH adjuster | polymer | Solvent |
| Example 24 | BBI-4000 | IBM* | HG* | Anhydrous | HPC | Anhydrous |
| | (5) | (2.5) | (10) | citric acid | (1.25) | ethanol |
| | | | | (0.05) | | (q.s. to 100**) |
| Example 25 | BBI-4000 | IPM | HG | Anhydrous | HPC | Anhydrous |
| | (15) | (2.5) | (10) | citric acid | (1.25) | ethanol |
| | | | | (0.05) | | (q.s. to 100) |
| Comparative | BBI-4000 | IPM | HG | Anhydrous | HPC | Anhydrous |
| Examples 5 and 6 | (5) | (2.5) | (10) | citric acid | (1.25) | ethanol |
| | | | | (0.001) | | (q.s. to 100) |

*IPM: Isopropyl myristate, HG: Hexylene glycol,.
**q.s. to 100: The reminder was constituted with anhydrous ethanol so that the total amount should be 100%.

### <Test Method>

The pH measurement method and viscosity measurement method were the same as in Test Example 1.

<Stability Test Method 1: Storage for 24 Months Under 25±2° C./60±5% RH with Light Shielding>

The formulations of Examples 24 and 25 were used for the stability test 1. The pH values mentioned in the tables mentioned below were the highest values during the storage period (i.e., up to the 24th month from the preparation of the formulations). The criteria for Judgment mentioned in the table were the same as in Test Example 1. The formulation of Comparative Example 5 was stored for 6 months under the same conditions.

<Stability Test Method 2: Storage for 12 Months Under 30±2° C./60±5% RH with Light Shielding>

The formulation of Comparative Example 6 was used for the stability test 2. The pH values mentioned in the table mentioned below were the highest values during the storage period (i.e., up to the 12th month from the preparation of the formulations). The criteria for Judgment mentioned in the table were the same as in Test Example 1.

TABLE 21

| | | | | | Viscosity | | |
|---|---|---|---|---|---|---|---|
| | | | | | increase/decrease rate | | |
| | | | | | compared with viscosity | | |
| | | | | | at the time of | | |
| | | Viscosity (mPa · s) | | | preparation (%) | | |
| | | At the time of | After 12 | After 24 | After 12 | After 24 | |
| Formulation | pH | preparation | months | months | months | months | Judgment |
| Example 24 | 4.1 | 459 | 459 | 482 | 0% | 5.0% | A |
| Example 25 | 3.9 | 737 | 723 | 737 | −1.9% | 0% | A |
| Comparative | 6.1 | 412 | 98 | No data | −76% | No data | B |
| Example 6 | | | | | | | |

TABLE 22

| | Ingredient and concentration | | | | | |
|---|---|---|---|---|---|---|
| | (w/w %) | | pH | | | |
| | Active | | At the time of | After 6 | After 12 | After 24 |
| Formulation | ingredient | pH adjuster | preparation | months | months | months |
| Example 24 | BBI-4000 | Anhydrous | 3.5 | 3.7 | 3.8 | 4.1 |
| | (5) | citric acid | | | | |
| | | (0.05) | | | | |
| Example 25 | BBI-4000 | Anhydrous | 3.4 | 3.6 | 3.6 | 3.9 |
| | (15) | citric acid | | | | |
| | | (0.05) | | | | |
| Comparative | BBI-4000 | Anhydrous | 5.9 | 5.4 | No data | No data |
| Example 5 | (5) | citric acid | | | | |
| | | (0.001) | | | | |
| Comparative | BBI-4000 | Anhydrous | 6.1 | No data | 4.9 | No data |
| Example 6 | (5) | citric acid | | | | |
| | | (0.001) | | | | |

The formulations of Comparative Examples 5 and 6 containing anhydrous citric acid at a concentration of 0.001 w/w % showed a pH value in the range of 6.1 to 5.9 at the time of the preparation, and when they were stored at room temperature, the pH values thereof varied over time, like the formulations of Comparative Examples 1 to 4 mentioned in Test Example 1.

The formulation of Comparative Example 6 containing anhydrous citric acid at a concentration of 0.001 w/w % showed a marked decrease in viscosity (−76%) after storage of 12 months at room temperature. On the other hand, the pH values of the formulations of Examples 24 and 25 containing anhydrous citric acid at a concentration of 0.05 w/w % were maintained at 5.2 or lower after the preparation of the formulations, and the change in viscosity over time was slight.

Therefore, if pH value of a sofpironium bromide formulation is maintained at 5.2 or lower after the preparation of the formulation, decrease in viscosity thereof over time can be suppressed.

In particular, it was revealed by this example that when formulations are stored at room temperature, formulations showing a pH value of 2.5 to 5.2 at any point up to the 6th month from the preparation of the formulations are preferred. For example, formulations showing a pH value in the range of 2.5 to 5.2 at a point after 1, 3, or 6 months from the preparation of the formulations are preferred.

### <Purity Test>

In the long-term storage test (25±2° C./60±5% RH with light shielding) of the formulations of Example 24 and Example 25, purity test (related substances) was conducted until the 24th month from the preparation of the formulations. The formulation of Example 24 showed a very small HPLC peak of ethyl cyclopentylmandelate in the 24th month. The formulation of Example 25 showed neither increase in related substance nor appearance of new related substance during the storage period after the preparation of the formulation. These results indicated that the formulations of Example 24 and 25 were stable in the period of the long-term storage test.

As described above, it was revealed as shown by Examples 24 and 25 that sofpironium bromide formulations of which pH value is maintained at 5.2 or lower show no increase in impurities over a long period of time, and decrease in viscosity over time of such formulations is suppressed, regardless of the storage temperature and other test conditions.

### Test Example 7

Verification Study of BBI-4000 in Subjects with Primary Axillary Hyperhidrosis

By a randomized, double-blind, parallel-group comparison in patients with primary axillary hyperhidrosis, superiority of the efficacy of a pharmaceutical formulation, which contained sofpironium bromide for external application (5 w/w % of sofpironium bromide, 1.25 w/w % of hydroxypropylcellulose, 2.5 w/w % of isopropyl myristate, 0.05 w/w % of anhydrous citric acid, 10 w/w % of hexylene glycol, and anhydrous ethanol as the remainder) and was applied to the axillae once daily before bedtime for 6 weeks, was verified as compared with a placebo formulation (0 w/w % of sofpironium bromide). The primary evaluation item was the percentage of test subjects showing an HDSS score of 1 or 2 at the end of the treatment and a ratio of total sweat weight in both axillae at the end of the treatment to the baseline (sweat weight measured before the treatment) of 0.5 or lower.

In this study, “before treatment” refers to a time point before the treatment is performed by the administration of the pharmaceutical formulation of sofpironium bromide.

In this study, “at the end of the treatment” refers to a time point of the visiting to the hospital that was the basis of the end of the treatment. The end of the treatment consists of three visiting days after a given administration period, and the HDSS score and sweat weight at the end of the treatment are represented with their median values, unless especially noted.

In this study, “during the treatment period” refers to the period between the start of the treatment and the end of the treatment.

In this study, “baseline” refers to each value concerning degree of symptoms measured prior to the administration and serving as the standard. The baseline is measured during a specified period of time prior to the administration.

The baseline HDSS score and sweat weight used in this study refer to the medians of measured values of the items on the days of three visits within 9 days, which are defined as the baseline 1, baseline 2, and baseline 3, respectively.

The number of days of the administration of the pharmaceutical formulation of sofpironium bromide is the number of days counted from the day of the baseline 3, which is taken as day 1, and the same shall apply to such expressions as “administration period” and “number of weeks of administration”. The day of the baseline 3 is the day on which the administration of the pharmaceutical formulation of sofpironium bromide is started.

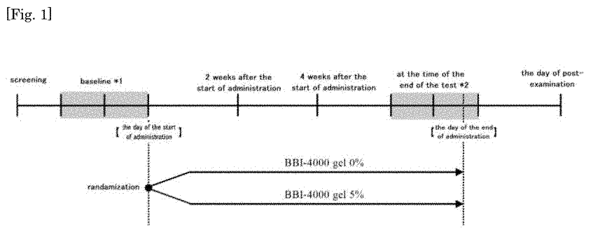

The time course of this test is shown in FIG. 1.

### <Analysis of Efficacy>

### (1) Primary Analysis of Efficacy

The analysis of the percentage of the subjects showing an HDSS score of 1 or 2 at the end of the treatment and a ratio of the total sweating weight of both axillae at the end of the treatment to the baseline of 0.5 or lower was performed by the chi-square test.

### (2) Secondary Analysis of Efficacy

### 1) Sweating Weight

The median of the total sweating weights in both axillae at the baselines 1 to 3 was defined as the baseline sweating weight, and the median of the total sweating weights in both axillae at 6th week of the administration 1 to 3 was defined as the total sweating weight in both axillae at the end of the treatment.

The basic statistics for the total sweating weight of both axillae were calculated for each treatment group and each test implementation time, and compared between the treatment groups. In addition, the following items were also calculated, and confidence intervals were given for differences between the treatment groups to perform statistical tests.
Percentage of the subjects with a ratio of total sweating weight in both axillae at the end of the treatment to the baseline of 0.5 or lower.
Change from the baseline in total sweating weight in both axillae at the end of the treatment.

### 2) HDSS

The median HDSS score at the baselines 1 to 3 was defined as the baseline HDSS score, and the median HDSS score of HDSS scores at the 6th week of administration 1 to 3 was defined as the HDSS score at the end of the treatment. The data were tabulated for each treatment group and each test implementation time. The percentage of the subjects with an HDSS score of 1 or 2 at the end of the treatment was also calculated, and confidence intervals were given for differences between treatment groups to perform statistical tests.

### <Efficacy Study Items>

The following items were investigated, and the results were recorded.

### (1) Sweating Weight Measurement

### 1) Measurement Conditions

Temperature: 20 to 28° C., humidity: 20 to 80% RH

### 2) Measurement Method

Pre-weighed filter paper was placed on both axillae of the subject for 5 minutes.

The weight of the filter paper containing sweat was then measured, and the sweating weight was calculated.

The measurement was performed for each subject at such a time in the period of from 8:00 a.m. to 7:00 p.m. that the difference of the test implementation times did not exceed 4 hours.

### (2) HDSS

The criteria for judgment of HDSS scores were as follows.

TABLE 23

| Score | Subjective symptoms |
|---|---|
| 1 | My sweating is never noticeable and never interferes with my daily |
| | activities. |
| 2 | My sweating is tolerable but sometimes interferes with my daily |
| | activities. |
| 3 | My sweating is barely tolerable and frequently interferes with my |
| | daily activities. |
| 4 | My sweating is intolerable and always interferes with my daily |
| | activities. |

### <Subject Patients and Major Inclusion Criteria>

Patients with primary axillary hyperhidrosis who were 12 years old or older and meet the following diagnostic criteria and conditions at the time of obtaining informed consent

1. Patients diagnosed with primary axillary hyperhidrosis who met at least 2 of the following 6 criteria during the screening interview.
(1) Symptoms first appeared at the age of 25 or younger.
(2) Symmetrical sweating is observed on both sides of the body.
(3) Sweating stops during sleep.
(4) One or more hyperhidrosis episodes per week.
(5) A family history of hyperhidrosis.
(6) Excessive sweating interferes with daily life.
2. Patients who meet all of the following conditions.
(1) HDSS score is 3 or 4 at each of time points of the baselines 1 to 3.
(2) Sweating weight in each axilla is 50 mg or more at any 2 of the 3 time points of the baselines 1 to 3.

### <Major Exclusion Criteria>

1. Patients with secondary hyperhidrosis.
2. Patients whose hyperhidrosis started or worsened with menopause.
3. Patients who are candidates for thoracic sympathectomy.

### <Patients for Clinical Trial>

The test medicaments were randomly assigned to 281 primary axillary hyperhidrosis patients (the 0% group consists of 140 patients, and the 5% group consists of 141 patients), and data of these patients as the subjects were analyzed.

### <Results for Primary Evaluation Item for Efficacy>

The percentages of the subjects with an HDSS score of 1 or 2 at the end of the treatment and a ratio of the total sweating weight of both axillae at the end of the treatment to the baseline of 0.5 or lower are shown in the table mentioned below.

TABLE 24

| | | | | |
|---|---|---|---|---|
| Administration | 0% BBI-4000 | (140) | 5% BBI-4000 | (141) |
| group (Number | | | | |
| of subjects) | | | | |
| Percentage | 36.4% | (51) | 53.9% | (76) |
| (Number of | | | | |
| subjects) | | | | |

| | | | |
|---|---|---|---|
| Difference from | — | 17.5% | (6.02~28.93) |
| the 0% group | | | |
| (95% confidence | | | |
| interval) | | | |

| | | |
|---|---|---|
| p-Value of chi- | — | 0.003 |
| square test versus | | |
| the 0% group | | |

The percentages of the subjects for whom the efficacy was observed were 36.4% (51/140) in the 0% group, and 53.9% (76/141) in the 5% group, i.e., the percentage was 17.5% (95% confidence interval: 6.02 to 28.93) higher in the 5% group than in the 0% group, indicating a statistically significant difference between the administration groups (Chi-square test: p=0.003).

### <Results for Secondary Evaluation Items for Efficacy>

### (1) HDSS

The percentages of the subjects with an HDSS score of 1 or 2 at the end of the treatment were 47.9% (67/140) in the 0% group and 60.3% (85/141) in the 5% group, i.e., the percentage was 12.4% (95% confidence interval: 0.86 to 23.99) higher in the 5% group than in the 0% group, indicating a statistically significant difference between the groups (Chi-square test: p=0.036).

### (2) Sweating Weight

The percentages of the subjects with a ratio of the total sweating weight in both axillae at the end of the treatment to the baseline of 0.5 or lower were 66.4% (93/140) in the 0% group and 77.3% (109/141) in the 5% group, i.e., the percentage was 10.9% (95% confidence interval: 0.44 to 21.32) higher in the 5% group than in the 0% group, indicating a statistically significant difference between the groups (Chi-square test: p=0.042).

<Results of Efficacy in Patients with Total Sweating Weight in Both Axillae of 400 mg or Greater>

In order to examine the efficacy in patients with more severe sweating, i.e., higher baseline total sweating weight in both axillae, the efficacy was examined for a subpopulation of the patients with a baseline total sweating weight in both axillae of 400 mg or greater.

The analysis results are shown in the table mentioned below.

TABLE 25

| | | | Percentage of subjects with | | | | | |
|---|---|---|---|---|---|---|---|---|
| | | | HDSS score at the end of | | | | | |
| | | | treatment of 1 or 2 and ratio of | | | | | |
| | | | total sweating weight in both | | | Percentage of subjects with | | |
| | Number of | | axillae at the end of treatment | | | HDSS score of 1 or 2 at the end | | |
| | subjects | | to baseline of 0.5 or smaller | | | of treatment | | |
| | 0% | 5% | 0% | 5% | Difference | 0% | 5% | Difference |
| Category | group | group | group | group | between groups | group | group | between |
| 100 mg <=, | 130 | 128 | 39.20% | 54.70% | 15.50% | 50.80% | 61.70% | 10.90% |
| <400 mg | | | | | | | | |
| 400 mg <= | 10 | 13 | 0.00% | 46.20% | 46.20% | 10.00% | 46.20% | 36.20% |

| | | | Percentage of subjects with | | | | |
|---|---|---|---|---|---|---|---|
| | | | ratio of total sweating weight in | | | | |
| | | | both axillae at the end of | | | | |
| | Number of | | treatment to basehne of 0.5 or | | | Mean total sweating weight in | |
| | subjects | | smaller | | | both axillae at the end of | |
| | 0% | 5% | 0% | 5% | Difference | treatment | |
| Category | group | group | group | group | between groups | 0% group | 5% group |
| 100 mg <=, | 130 | 128 | 67.70% | 76.60% | 8.90% | 83.3 mg | 60.7 mg |
| <400 mg | | | | | | | |
| 400 mg <= | 10 | 13 | 50.00% | 84.60% | 34.60% | 301.1 mg | 167.7 mg |

The major evaluation item, the “percentage of subjects with an HDSS score of 1 or 2 at the end of the treatment and a ratio of total sweating weight in both axillae at the end of the treatment to the baseline of 0.5 or lower”, was higher in the 5% group than in the 0% group for both categories. The difference between groups was 15.5% in the 100 mg or higher and lower than 400 mg category, and 46.2% in the 400 mg or higher category. That is, the difference between groups was greater in the 400 mg or higher category.

The “percentage of subjects with an HDSS score of 1 or 2 at the end of the treatment” and the “percentage of subjects with a ratio of total sweating weight in both axillae at the end of the treatment to the baseline of 0.5 or smaller” were higher in the 5% group than in the 0% group for the both categories. The mean total sweating weight in both axillae was lower in the 5% group than in the 0% group for the both categories at any evaluation point after the administration. At the end of the treatment, the mean total sweating weight in both axillae was lower in the 5% group than in the 0% group for the both categories.

As described above, better amelioration was observed in the 5% group than in the 0% group for the both evaluation items in the subjects with total sweating weight in both axillae of 400 mg or larger.

### <Change in HDSS Score Before and After Treatment in the 5% BBI-4000 Group>

In a randomized, double-blind, parallel-group comparison for patients with primary axillary hyperhidrosis, the difference (ΔHDSS) of each subject's HDSS scores before the treatment, consisting of application of 5% BBI-4000 to the axillae once daily for 6 weeks, and at the end of the treatment was calculated by subtracting the latter from the former. The 140 cases for which both the HDSS scores before the treatment and at the end of the treatment were obtained were included in the analysis, and the mean change and standard deviation of ΔHDSS were calculated.

As a result, the mean ΔHDSS in the 5% BBI-4000 group was 1.14±0.87.

### INDUSTRIAL APPLICABILITY

According to the present invention, as a non-aqueous and low water content sofpironium bromide formulation, a stable composition in which the decrease in viscosity over time during long-term storage is suppressed can be provided.

Furthermore, the formulation of the present invention can be used for medical care, therapeutic treatment, or prevention of primary axillary hyperhidrosis.

1. A pharmaceutical formulation for applying as an external application to the surface of the human body, wherein the formulation contains:
(a) sofpironium bromide,
(b) one or more kinds of water-soluble polymers, and
(c) ethanol,
wherein the formulation has a pH of 5.2 or lower and is a uniformly dispersed, and non-aqueous formulation or low water content formulation with a water content of 5 w/w % or lower, and
wherein the pH is measured at any one or more time points selected within 6 months after preparation of the formulation, the pH is determined by measuring pH of the formulation stored at room temperature, and the pH is a value obtained after a pH electrode for non-aqueous solvent is immersed in the formulation for 5 minutes.

2. The formulation according to claim 1, wherein content of sofpironium bromide is 1 to 20 w/w % based on the total weight of the formulation.

3. The formulation according to claim 1, wherein the pH is in the range of 2.5 to 5.2.

4. The formulation according to claim 1, wherein ingredients in the formulation are uniformly dissolved.

5. The formulation according to claim 1, wherein the water-soluble polymer or polymers consist of a water-soluble vinyl polymer or a water-soluble cellulose polymer.

6. The formulation according to claim 1, wherein the water-soluble polymer or polymers are selected from the group consisting of hydroxymethylcellulose, hydroxyethylcellulose, hydroxypropylcellulose, hydroxyethylmethylcellulose, hydroxypropylmethylcellulose, methylcellulose, ethylcellulose, carboxymethylcellulose, a carboxyvinyl polymer, polyvinyl alcohol, a polyvinyl copolymer, polyvinylpyrrolidone, and copovidone.

7. The formulation according to claim 1, wherein the water-soluble polymer or polymers consist of hydroxypropylcellulose or a carboxyvinyl polymer.

8. The formulation according to claim 1, wherein content of the water-soluble polymer or polymers is 0.01 to 5.0 w/w % based on the total weight of the formulation.

9. The formulation according to claim 1, wherein content of ethanol is 50 to 99 w/w % based on the total weight of the formulation.

10. The formulation according to claim 1, further containing a pH adjuster.

11. The formulation according to claim 10, wherein the pH adjuster is an acid selected from the group consisting of tartaric acid, acetic acid, and citric acid, or a salt thereof.

12. The formulation according to claim 10, wherein the amount of the pH adjuster is 0.015 to 5 w/w % based on the total weight of the formulation.

13. The formulation according to claim 1, further containing a non-volatile oil (except for formulation containing Dimethiconol Blend 20).

14. The formulation according to claim 13, wherein the non-volatile oil is selected from the group consisting of a non-volatile ester, a non-volatile ether, a non-volatile silicone, and a non-volatile alcohol.

15. The formulation according to claim 13, wherein the non-volatile oil is a non-volatile ester selected from the group consisting of a monoester, a diester, and a trimester, and wherein the non-volatile oil is represented as R₁COOR₂, wherein one of R₁ and R₂ is a C₄-C₄₀ straight chain alkyl group that may be substituted, or a C₄-C₄₀ branched chain alkyl group that may be substituted, and the other of R₁ and R₂ is a C₁-C₄₀ alkyl group that may be substituted.

16. The formulation according to claim 13, wherein the non-volatile oil is a non-volatile fatty acid ester selected from the group consisting of ethyl myristate, 2-octyldodecyl myristate, butyl stearate, isocetyl stearate, 2-octyldodecyl stearate, hexyl laurate, 2-hexyldecyl laurate, 2-ethylhexyl palmitate, 2-octyldecyl palmitate, cetearyl octanoate, isononyl isononanoate, octyldodecyl neopentanoate, 2-octyldodecyl erucate, 2-octyldodecyl benzoate, a decanoic acid ester, a ricinoleic acid ester, isopropyl myristate, diisopropyl adipate, a medium chain fatty acid triglyceride, isopropyl palmitate, an alkyl (C₁₄-C₁₈) ethylhexanoate, myristyl myristate, ethyl oleate, oleyl oleate, ethylhexyl palmitate, cetyl palmitate, 2-hexyldecyl myristate, 2-hexyldecyl palmitate, PPG-3 benzyl ether myristate, isotridecyl isononanoate, triethylhexyl trimellitate, an alkyl (C₁₂-C₁₅) benzoate, diethoxyethyl succinate, propylene glycol dicaprate, propylene glycol dicaprylate, glyceryl tri(caprylate/caprate), triethylhexanoin, triisostearin, isopropyl isostearate, isostearyl isostearate, polyglyceryl-2 triisostearate, diethylhexyl succinate, PPG-2 myristyl propionate, pentaerythrityl tetraisostearate, diethyl sebacate, PPG-3 benzyl ether ethylhexanoate, glyceryl tribehenate, cetyl 2-ethylhexanoate, diisostearyl malate, 2-ethylhexyl stearate, triethylhexyl citrate, and an alkyl lactate such as ethyl lactate.

17. The formulation according to claim 13, wherein the non-volatile oil is a non-volatile fatty acid ester selected from the group consisting of isopropyl myristate, diisopropyl adipate, and a medium chain fatty acid triglyceride.

18. The formulation according to claim 13, wherein the non-volatile oil is a non-volatile silicone selected from the group consisting of a medical grade silicone oil, methylphenyl silicone, methyl terminated hydrogen branched silicone, decamethyl pentacyclosiloxane, octamethyl tetracyclosiloxane, cyclomethicone 5-NF, PEG-12 dimethicone, dimethicone 20 cSt, dimethicone 100 cSt, dimethicone 350 cSt, dimethicone 500 cSt, dimethicone 1000 cSt, and dimethicone 12500 cSt.

19. The formulation according to claim 13, wherein the non-volatile oil is a non-volatile silicone selected from the group consisting of cyclomethicone 5-NF, PEG-12 dimethicone, dimethicone 20 cSt, and dimethicone 350 cSt.

20. The formulation according to claim 13, wherein content of the non-volatile oil is 0.5 to 10 w/w % based on the total weight of the formulation.

21. The formulation according to claim 1, further containing a polyhydric alcohol.

22. The formulation according to claim 21, wherein the polyhydric alcohol is selected from the group consisting of hexylene glycol, propylene glycol, ethylene glycol, glycerol, and butylene glycol.

23. The formulation according to claim 21, wherein content of the polyhydric alcohol is 1.0 to 30 w/w % based on the total weight of the formulation.

24. The formulation according to claim 1, wherein viscosity of the formulation is 10 to 2000 mPa·s at 25° C.

25. The formulation according to claim 1, wherein viscosity of the formulation is 10 to 1000 mPa·s at 25° C. after storage at room temperature for 36 months from preparation of the formulation or after storage at 40° C. for 3 months from preparation of the formulation.

26. The formulation according claim 1, which is for medical care, therapeutic treatment, or prevention of a disease selected from the group consisting of hyperhidrosis, overactive bladder, chronic obstructive pulmonary disease, cardiac disease, salivation, ocular disease, and bronchial asthma.

27. The formulation according to claim 1, wherein content of a compound (II) represented by the following formula (II): 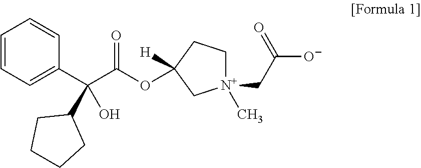 [SMILES: C[N@@+]1(CC(=O)[O-])CC[C@@H](OC(=O)[C@](O)(c2ccccc2)C2CCCC2)C1]
and epimer at N⁺
is 1.5 w/w % or lower based on the content of sofpironium bromide, and purity of sofpironium bromide is 90 w/w % or higher after storage at room temperature for 36 months from preparation of the formulation, or after storage at 40° C. for 3 months from preparation of the formulation.
